SEQUENCE LISTING (7 sequences)

SEQ ID NO: 1 - protein, 324 aa, Artificial Sequence
  GpD-ASPH-1lambda fusion protein
HMTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIVGGSGPVGPGGSGASSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 2 - protein, 111 aa, Bacteriophage lambda
HMTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIV

SEQ ID NO: 3 - protein, 14 aa, Artificial Sequence
  linker sequence
GGSGPVGPGGSGAS

SEQ ID NO: 4 - protein, 199 aa, Homo sapiens
STSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 5 - protein, 314 aa, Homo sapiens
DRAMAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 6 - protein, 176 aa, Homo sapiens
LDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDP

SEQ ID NO: 7 - protein, 238 aa, Homo sapiens
GTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETRTWEEGKVLIFDDSFEHEVWQDASSFRLIFIVDVWHPELTPQQRRSLPAI

(19) United States
(12) Patent Application Publication
(10) Pub. No.: US 2020/0345820 A1
(43) Pub. Date: Nov. 5, 2020

(54) COMBINATION THERAPIES FOR CANCER

(71) Applicant: SENSEI BIOTHERAPEUTICS, INC., Gaithersburg, MD (US)

(72) Inventors: Hossein A. Ghanbari, Potomac, MD (US); Ildiko Csiki, Glendora, CA (US)

(21) Appl. No.: 16/864,641

(22) Filed: May 1, 2020

Related U.S. Application Data
(60) Provisional application No. 62/841,500, filed on May 1, 2019.

(51) Int. Cl. A61K 38/50; A61K 35/76; A61P 35/00; A61K 9/00; A61K 39/395

(52) U.S. Cl. CPC A61K 38/50; A61K 35/76; A61K 39/3955; A61P 35/00; A61K 9/0021; C12Y 305/01

(57) ABSTRACT

Disclosed herein are methods for treating cancer by administering to a subject a composition comprising a bacteriophage expressing a fragment of human aspartate β-hydroxylase (ASPH) and an immune checkpoint protein inhibitor.

### CROSS-REFERENCE TO RELATED APPLICATIONS

This application claims priority to U.S. Provisional Patent Application No. 62/841,500, filed on May 1, 2019. The disclosure of this application is incorporated herein by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SEBI_020_001US_SeqList_ST25.txt, date recorded: May 1, 2020, file size ˜12,554 bytes).

### TECHNICAL FIELD

The disclosure relates to cancer immunotherapy, in particular to combinations of a tumor-associated antigen vaccine and an immune checkpoint protein inhibitor.

### BACKGROUND

Aspartate β-hydroxylase (ASPH) is a type II transmembrane protein predominantly expressed during embryogenesis, where it promotes cell migration for organ development. ASPH has very low expression in healthy adult tissue, and is localized to the intracellular compartment of the endoplasmic reticulum. However, re-expression and translocation to the tumor cell surface has been detected in more than 20 different types of cancers including lung, liver, colon, pancreas, prostate, ovary, bile duct, and breast cancers, with expression levels inversely correlated with disease prognosis (Yeung et al., (2007) Human Antibodies, 16, 163-176). Combination immunotherapeutic approaches targeting ASPH are proposed to treat cancer.

### SUMMARY

Provided herein is a method for inhibiting growth and/or proliferation of cancer cells in a subject, the method comprising administering to the subject an effective amount of a composition comprising a bacteriophage expressing a fragment of human aspartate β-hydroxylase (ASPH) and an effective amount of an immune checkpoint protein inhibitor. In some embodiments, the cancer cells are prostate, liver, bile duct, brain, head-and-neck, breast, colon, ovarian, cervical, pancreatic or lung cancer cells. In some embodiments, the cancer cells express human ASPH.

Further provided herein is a method for treating or ameliorating cancer or a symptom of cancer in a subject, the method comprising administering to the subject an effective amount of a composition comprising a bacteriophage expressing a fragment of human aspartate β-hydroxylase (ASPH) and an effective amount of an immune checkpoint protein inhibitor. In some embodiments, the cancer is prostate, liver, bile duct, brain, head-and-neck, breast, colon, ovarian, cervical, pancreatic or lung cancer. In some embodiments, the cancer is ASPH-positive squamous cell cancer of the head and neck (SCCHN). In some embodiments, the SCCHN is locally advanced unresectable SCCHN, metastatic SCCHN or recurrent SCCHN. In some embodiments, the cancer is a hematologic malignancy. In some embodiments, the cancer is human ASPH-expressing cancer.

In some embodiments of the methods and compositions provided herein, the bacteriophage is bacteriophage lambda. In some embodiments, the bacteriophage lambda expresses amino acids 113-311 from the N-terminal region of ASPH fused at the C-terminus of the bacteriophage lambda head decoration protein D (gpD). In some embodiments, the bacteriophage lambda expresses a fusion protein comprising, in N-terminus to C-terminus order, (1) a gpD fragment, (2) a linker sequence and (3) a fragment of human ASPH. In some embodiments, the gpD fragment is the amino acid sequence of SEQ ID NO: 2. In some embodiments, the linker sequence comprises SEQ ID NO: 3. In some embodiments, the fragment of human ASPH consists of SEQ ID NO: 4. In some embodiments, the bacteriophage lambda expresses a protein comprising or consisting of the amino acid sequence of SEQ ID NO:1.

In some embodiments of the methods provided herein, the composition comprising a bacteriophage is administered at a dose of about 1×10¹¹ particles in a 1 ml intradermal injection. In some embodiments, the composition comprising a bacteriophage is administered every 3 weeks ±3 days for 4 doses, then every 6 weeks ±3 days for 6 additional doses, and thereafter every 12 weeks ±3 days for up to 24 months.

In some embodiments of the methods provided herein, the immune checkpoint protein is Programmed Death-1 (PD-1) or Programmed Death Ligand-1 (PD-L1). In some embodiments, the immune checkpoint protein inhibitor disrupts the interaction between PD-1 and PD-L1. In some embodiments, the immune checkpoint protein inhibitor is an antibody or an antibody fragment that targets PD-1. In some embodiments, the antibody that targets PD-1 is pembrolizumab. In some embodiments, the pembrolizumab is administered at a dose of about 200 mg as an intravenous infusion over about 30 minutes. In some embodiments, the pembrolizumab is administered about every 3 weeks. In some embodiments, the immune checkpoint protein inhibitor is an antibody or an antibody fragment that targets PD-L1.

Provided herein is a method for treating or ameliorating cancer or a symptom of cancer in a subject, the method comprising administering to the subject an effective amount of a composition comprising a bacteriophage expressing a fragment of human ASPH and an effective amount of an immune checkpoint protein inhibitor; wherein the composition comprising the bacteriophage comprises bacteriophage lambda expressing a fusion protein comprising or consisting of the amino acid sequence of SEQ ID NO: 1; wherein the composition comprising the bacteriophage is administered at a dose of about 1×10¹¹ particles in a 1 ml intradermal injection every 3 weeks ±3 days for 4 doses then every 6 weeks ±3 days for 6 additional doses, thereafter every 12 weeks ±3 days; and wherein the immune checkpoint protein inhibitor is pembrolizumab, and wherein the pembrolizumab is administered at a dose of about 200 mg as an intravenous infusion over about 30 minutes every 3 weeks. In some embodiments, the cancer is ASPH-positive head-and-neck cancer.

Also provided herein is a composition comprising: a pharmaceutical composition comprising a bacteriophage expressing a fragment of human aspartate β-hydroxylase (ASPH); and a pharmaceutical composition comprising an immune checkpoint protein inhibitor; wherein said pharmaceutical compositions are in separate containers. In some embodiments, the bacteriophage is a bacteriophage lambda that expresses a protein comprising or consisting of the amino acid sequence of SEQ ID NO:1. In some embodiments, the immune checkpoint protein is Programmed Death-1 (PD-1) or Programmed Death Ligand-1 (PD-L1).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the amino acid sequence (SEQ ID NO:1) of the GpD-ASPH-1λ fusion protein. The sequence portions shown in N-terminus to C-terminus order are: (1) gpD fragment sequence (SEQ ID NO: 2), (2) linker sequence (SEQ ID NO: 3); and (3) ASPH fragment sequence (SEQ ID NO: 4).

FIG. 2 is a schematic depicting an experimental protocol for studying effects of treatment with (1) a composition comprising a bacteriophage expressing a fragment of human ASPH, (2) an anti-PD-1 antibody or (3) a combination of a composition comprising a bacteriophage expressing a fragment of human ASPH in an animal model of hepatocellular carcinoma (HCC). “Vaccine” or “vac” refers to treatment with a bacteriophage lambda nanoparticle vaccine, wherein the bacteriophage lambda expresses a protein having the amino acid sequence of SEQ ID NO: 1. “PD-1 blockade” refers to treatment with the anti-mouse PD-1 (CD279) monoclonal antibody (InVivoMAb; Clone: RMP1-14). pfu=plaque forming units. i.p.=intraperitoneal. BNL=BNL 1ME A.7R.1 HCC cells (ATCC).

FIG. 3A depicts photographs of subcutaneous tumors formed by BNL cells in Balb/c mice at day 43 after tumor inoculation. FIG. 3B is a line graph depicting tumor volume (mm³) in mice over time after tumor inoculation with BNL cells. “control” refers to IgG antibody. “PD-1 & vaccine” refers to treatment with an anti-mouse PD-1 (CD279) monoclonal antibody and a bacteriophage lambda nanoparticle vaccine expressing a protein having the amino acid sequence of SEQ ID NO: 1.

FIG. 4A depicts photographs of subcutaneous tumors formed by BNL cells in Balb/c mice at day 43 after tumor inoculation. FIG. 4B is a line graph depicting tumor volume (mm³) in mice over time after tumor inoculation with BNL cells. “control” refers to IgG antibody. “PD-1” refers to treatment with an anti-mouse PD-1 (CD279) monoclonal antibody. “vaccine” refers to treatment with a bacteriophage lambda nanoparticle vaccine expressing a protein having the amino acid sequence of SEQ ID NO: 1. “PD-1 & vaccine” refers to treatment with an anti-mouse PD-1 (CD279) monoclonal antibody and a bacteriophage lambda nanoparticle vaccine expressing a protein having the amino acid sequence of SEQ ID NO: 1.

FIG. 5 is a line graph depicting representative data from an in vitro cytotoxicity assay of specific treatments against BNL cells. “Anti-PD-1+ASPH vaccine” refers to treatment with an anti-mouse PD-1 (CD279) monoclonal antibody and a bacteriophage lambda nanoparticle vaccine expressing a protein having the amino acid sequence of SEQ ID NO: 1.

FIG. 6 is a line graph depicting representative data from an in vitro cytotoxicity assay of specific treatments against BNL cells. “Anti-PD-1” refers to treatment with an anti-mouse PD-1 (CD279) monoclonal antibody. “Vaccine” refers to treatment with a bacteriophage lambda nanoparticle vaccine expressing a protein having the amino acid sequence of SEQ ID NO: 1. “Anti-PD-1+Vaccine” refers to treatment with an anti-mouse PD-1 (CD279) monoclonal antibody and a bacteriophage lambda nanoparticle vaccine expressing a protein having the amino acid sequence of SEQ ID NO: 1.

FIG. 7 is a line graph depicting representative data from an in vitro cytotoxicity assay of specific treatments against BNL cells. The data were collected at day 4 of the assay. “Anti-PD-1” refers to treatment with an anti-mouse PD-1 (CD279) monoclonal antibody. “Vaccine” refers to treatment with a bacteriophage lambda nanoparticle vaccine expressing a protein having the amino acid sequence of SEQ ID NO: 1. “Anti-PD-1+Vaccine” refers to treatment with an anti-mouse PD-1 (CD279) monoclonal antibody and a bacteriophage lambda nanoparticle vaccine expressing a protein having the amino acid sequence of SEQ ID NO: 1.

FIG. 8 is a series of plots depicting representative data from flow cytometry studies of ASPH antigen specific T cell activation in a mouse BNL cell HCC model. “Anti-PD-1” refers to treatment with an anti-mouse PD-1 (CD279) monoclonal antibody. “ASPH-Phage Vaccine” refers to treatment with a bacteriophage lambda nanoparticle vaccine expressing a protein having the amino acid sequence of SEQ ID NO: 1. “Combination Therapy” refers to treatment with an anti-mouse PD-1 (CD279) monoclonal antibody and a bacteriophage lambda nanoparticle vaccine expressing a protein having the amino acid sequence of SEQ ID NO: 1. The phage vaccine increased T cell activation. In combination with anti-PD-1 antibody, the response was amplified.

FIG. 9A-FIG. 9B are bar graphs depicting representative data from studies measuring levels of anti-ASPH antibodies in serum of mice treated with different therapies. FIG. 9A shows results from a hepatocellular carcinoma (HCC) mouse model. FIG. 9B shows results from a breast cancer mouse model. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 10A-FIG. 10B depict representative data from studies of infiltration of hepatocellular carcinoma (HCC) tumors with CD3⁺ T cells at the end of therapy (7 weeks). “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 11 is a bar graph depicting representative data from an in vitro cytotoxicity assay of specific treatments against 4T1 murine breast cancer cells. “Vaccine & PD-1” refers to treatment with an anti-mouse PD-1 (CD279) monoclonal antibody and a bacteriophage lambda nanoparticle vaccine expressing a protein having the amino acid sequence of SEQ ID NO: 1.

FIG. 12 is a schematic depicting an experimental protocol for studying effects of treatment with (1) a composition comprising a bacteriophage expressing a fragment of human ASPH, (2) an anti-PD-1 antibody or (3) a combination of a composition comprising a bacteriophage expressing a fragment of human ASPH in an animal model of breast cancer. “Vaccine” refers to treatment with a bacteriophage lambda nanoparticle vaccine, wherein the bacteriophage lambda expresses a protein having the amino acid sequence of SEQ ID NO: 1. “PD-1 inhibitor injection” refers to treatment with the anti-mouse PD-1 (CD279) monoclonal antibody (InVivoMAb; Clone: RMP1-14). pfu=plaque forming units. 4T1=4T1 mammary carcinoma mouse cells (ATCC).

FIG. 13 is a line graph depicting tumor volume in mice over time after inoculation with 4T1 cells. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 14 is a set of photographs depicting representative examples of 4T1 breast tumor growth in mice after inoculation with 4T1 cells. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 15 is a bar graph depicting the number of pulmonary metastases in mice after inoculation with 4T1 cells. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 16 is a photograph depicting pulmonary metastases in mice after inoculation with 4T1 cells. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 17 is a series of photographs depicting representative examples of metastases in mice after inoculation with 4T1 cells and a table providing numbers of metastases in various organs. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 18 is a series of micrographs depicting metastatic spread of 4T1 breast cancer in mice after inoculation with 4T1 cells.

FIG. 19A-FIG. 19C depict representative data analyzing metastatic burden in mice after inoculation with 4T1 cells. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 20 is a line graph depicting representative data from an in vitro cytotoxicity assay of specific treatments against 4T1 murine breast cancer cells. “anti-PD-1” refers to treatment with an anti-mouse PD-1 (CD279) monoclonal antibody. “Vaccine” refers to treatment with a bacteriophage lambda nanoparticle vaccine expressing a protein having the amino acid sequence of SEQ ID NO: 1. “Combined” refers to treatment with an anti-mouse PD-1 (CD279) monoclonal antibody and a bacteriophage lambda nanoparticle vaccine expressing a protein having the amino acid sequence of SEQ ID NO: 1.

FIG. 21 is a series of plots depicting representative data from flow cytometry studies of ASPH antigen specific T cell activation in a mouse 4T1 breast tumor model. “ASPH-Phage Vaccine” refers to treatment with a bacteriophage lambda nanoparticle vaccine expressing a protein having the amino acid sequence of SEQ ID NO: 1. “anti-PD-1+ASPH-Phage vaccine” refers to treatment with an anti-mouse PD-1 (CD279) monoclonal antibody and a bacteriophage lambda nanoparticle vaccine expressing a protein having the amino acid sequence of SEQ ID NO: 1. The phage vaccine increased T cell activation. In combination with anti-PD-1 antibody, the response was amplified.

FIG. 22 is a series of plots depicting representative data from flow cytometry studies of ASPH antigen specific T cell activation in a mouse 4T1 breast tumor model. “ASPH-Phage Vaccine” refers to treatment with a bacteriophage lambda nanoparticle vaccine expressing a protein having the amino acid sequence of SEQ ID NO: 1. “anti-PD-1+ASPH-Phage vaccine” refers to treatment with an anti-mouse PD-1 (CD279) monoclonal antibody and a bacteriophage lambda nanoparticle vaccine expressing a protein having the amino acid sequence of SEQ II) NO: 1. The phage vaccine increased T cell activation. In combination with anti-PD-1 antibody, the response was amplified.

FIG. 23 is a line graph depicting tumor volume in mice over time after inoculation with 4T1 cells. Different amounts of a PD-1 inhibitor (anti-PD-1 antibody) were used to treat mice. The dose response curve showed that a 50% reduction in PD-1 inhibitor still produced a statistically significant reduction in tumor volume compared to the lowest dose.

FIG. 24 is a bar graph depicting representative data from studies measuring the number of pulmonary metastases in mice after inoculation with 4T1 cells. Mice were treated with varying amounts of an anti-mouse PD-1 (CD279) monoclonal antibody (PD-1 inhibitor) and a bacteriophage lambda nanoparticle vaccine expressing a protein having the amino acid sequence of SEQ ID NO: 1 (vaccine). There is a statistically significant difference in the number of metastatic lesions comparing the highest and lowest dose of anti-PD-1 antibody. When the anti-PD-1 antibody doses were 50% reduced to 100 μg and again to 50 μg, there was still a significant difference in the number of metastatic lesions. These results suggested that it is possible to reduce the dosage of PD-1 inhibitor and retain a significant anti-metastatic effect.

FIG. 25 is a series of plots depicting representative data from flow cytometry studies of ASPH antigen specific T cell activation in a mouse 4T1 breast tumor model. Mice were treated with varying amounts of an anti-mouse PD-1 (CD279) monoclonal antibody and a bacteriophage lambda nanoparticle vaccine expressing a protein having the amino acid sequence of SEQ ID NO: 1. This figure shows the effect of reducing the anti-PD-1 antibody dosage in the combination with the nanoparticle vaccine on the activation of T cells as evidenced by interferon gamma (IFN-γ) levels.

FIG. 26 is a series of plots depicting representative data from flow cytometry studies of ASPH antigen specific T cell activation in a mouse 4T1 breast tumor model. Mice were treated with varying amounts of an anti-mouse PD-1 (CD279) monoclonal antibody and a bacteriophage lambda nanoparticle vaccine expressing a protein having the amino acid sequence of SEQ ID NO: 1. This figure shows the effect of reducing the anti-PD-1 antibody dosage in the combination with nanoparticle vaccine on the activation of T cells as evidenced by CD154 and CD137 levels.

FIG. 27A-FIG. 27B depict representative data from studies of CD3⁺ lymphocytes in mammary tumors in a mouse 4T1 breast tumor model. FIG. 27A is a series of micrographs showing CD3⁺ cells in mammary tumor tissue. FIG. 27B is a bar graph depicting the number of CD3⁺ cells/5 fields in various treatment groups. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1. The combination of anti-PD-1 antibody and the nanoparticle vaccine resulted in a statistically significant increase in the number of tumor-infiltrating lymphocytes entering the tumor compared to the control.

FIG. 28A-FIG. 28B depict representative data from studies of CD3⁺ lymphocytes in pulmonary metastases in a mouse 4T1 breast tumor model. FIG. 28A is a series of micrographs showing CD3⁺ cells in pulmonary metastatic tumor tissue. FIG. 28B is a bar graph depicting the number of CD3⁺ cells/5 fields in various treatment groups. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1. The combination of anti-PD-1 antibody and the nanoparticle vaccine resulted in a statistically significant increase in the number of tumor-infiltrating lymphocytes entering the pulmonary metastasis tissue compared to the control.

FIG. 29A-FIG. 29D depict representative data from studies of CD8⁺ T cells in mammary tumors and pulmonary metastases in a mouse 4T1 breast tumor model. FIG. 29A is a series of micrographs showing CD8⁺ cells in mammary tumor tissue and pulmonary metastatic tumor tissue. FIG. 29B is a bar graph depicting the number of CD8⁺ cells in various treatment groups. FIG. 29C is a series of micrographs showing CD45RO cells in mammary tumor tissue and pulmonary metastatic tumor tissue. FIG. 29D is a bar graph depicting the number of CD45RO cells in various treatment groups. “PD-1 inhibitor group” refers to treatment with an anti-mouse PD-1 (CD279) monoclonal antibody. “Vaccine group” refers to treatment with a bacteriophage lambda nanoparticle vaccine expressing a protein having the amino acid sequence of SEQ ID NO: 1. “Combination group” refers to treatment with an anti-mouse PD-1 (CD279) monoclonal antibody and a bacteriophage lambda nanoparticle vaccine expressing a protein having the amino acid sequence of SEQ ID NO: 1.

FIG. 30 is a schematic of the clinical trial described in Example 2. “Q” stands for “every”.

### DETAILED DESCRIPTION

The disclosure provides combination therapies for treating cancer that encompass treatment with a bacteriophage expressing a fragment of human aspartate β-hydroxylase (ASPH, also known as HAAH) and an effective amount of an immune checkpoint protein inhibitor.

In some embodiments, the disclosure provides a method for inhibiting growth and/or proliferation of cancer cells in a subject, the method comprising administering to the subject an effective amount of a composition comprising a bacteriophage expressing a fragment of human aspartate β-hydroxylase (ASPH) and an effective amount of an immune checkpoint protein inhibitor. In some embodiments, the cancer cells are prostate, liver (e.g., hepatocellular carcinoma), bile duct, brain, head-and-neck, breast (e.g., triple negative breast cancer), colon, ovarian, cervical, pancreatic or lung cancer cells. In some embodiments, the cancer cells express human ASPH.

In some embodiments, the disclosure further provides a method for treating or ameliorating cancer or a symptom of cancer in a subject, the method comprising administering to the subject an effective amount of a composition comprising a bacteriophage expressing a fragment of human ASPH and an effective amount of an immune checkpoint protein inhibitor. The disclosure also provides a composition comprising a bacteriophage expressing a fragment of human ASPH and an immune checkpoint protein inhibitor for use in a method for treating or ameliorating cancer or a symptom of cancer in a subject. In some embodiments, the cancer is a solid tumor. In some embodiments, the cancer is a hematologic malignancy. In some embodiments, the cancer is prostate, liver (e.g., hepatocellular carcinoma), bile duct, brain, head-and-neck, breast (e.g., triple negative breast cancer), colon, ovarian, cervical, pancreatic or lung cancer. In some embodiments, the cancer is human ASPH-expressing cancer.

Illustrative examples of cancers that may be prevented, treated, or ameliorated with the methods and compositions disclosed herein include, but are not limited to, adenomas, carcinomas, sarcomas, leukemias, lymphomas, and multiple myelomas. In some embodiments, the cancer to be prevented, treated, or ameliorated with the methods and compositions disclosed herein is acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), B cell acute lymphocytic leukemia (B-ALL), chronic myelomonocytic leukemia (CMML) and polycythemia vera, Hodgkin lymphoma, nodular lymphocyte-predominant Hodgkin lymphoma and Non-Hodgkin lymphoma, including but not limited to, B-cell non-Hodgkin lymphomas: Burkitt lymphoma, small lymphocytic lymphoma (SLL), diffuse large B-cell lymphoma (DLBCL), follicular lymphoma, immunoblastic large cell lymphoma, precursor B-lymphoblastic lymphoma, and mantle cell lymphoma; and T-cell non-Hodgkin lymphomas: mycosis fungoides, anaplastic large cell lymphoma, Sézary syndrome, and precursor T-lymphoblastic lymphoma; overt multiple myeloma, smoldering multiple myeloma (MGUS), plasma cell leukemia, non-secretory myeloma, IgD myeloma, osteosclerotic myeloma, solitary plasmacytoma of bone, and extramedullary plasmacytoma, renal cell carcinoma (RCC), neuroblastoma, colorectal cancer, breast cancer, ovarian cancer, melanoma, sarcoma, prostate cancer, lung cancer (e.g., non-small cell lung cancer (NSCLC), small cell lung cancer or lung carcinoid tumor), esophageal cancer, hepatocellular carcinoma, pancreatic cancer, astrocytoma, mesothelioma, head-and-neck cancer, medulloblastoma or liver cancer. In some embodiments, the cancer is squamous cell cancer of the head and neck (SCCHN). In some embodiments, the cancer is ASPH-positive locally advanced unresectable or metastatic/recurrent SCCHN.

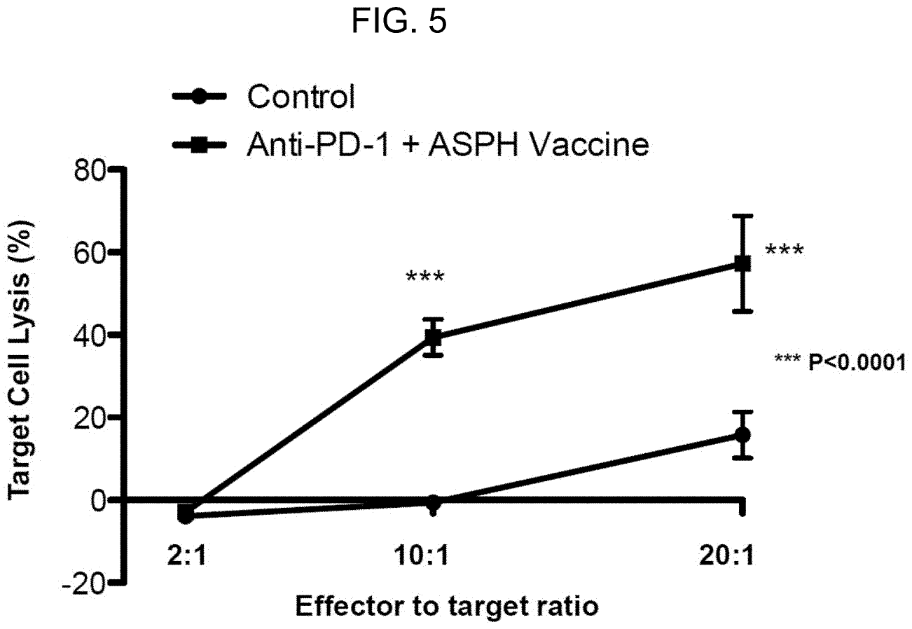

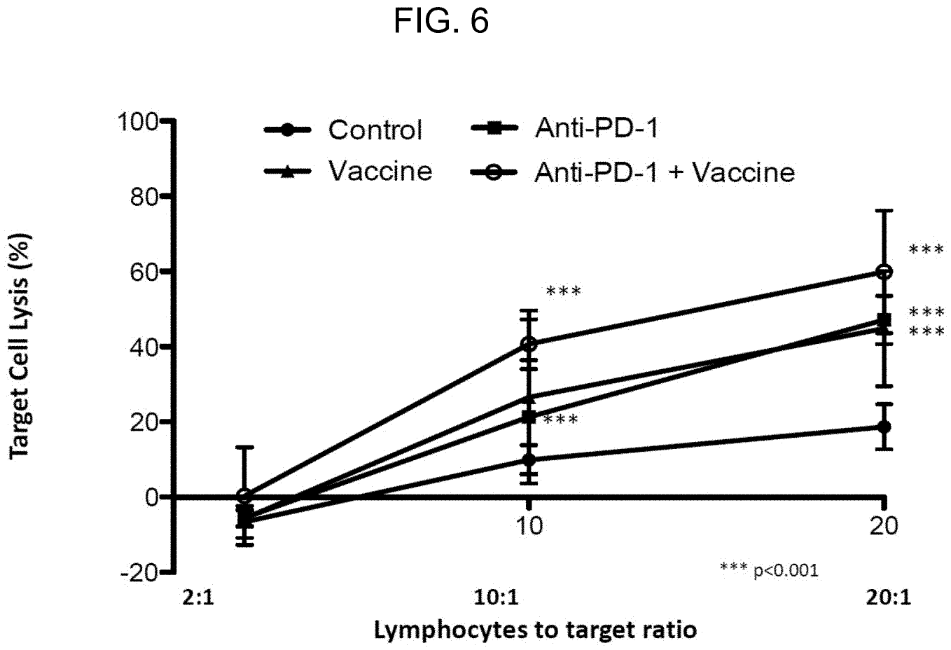

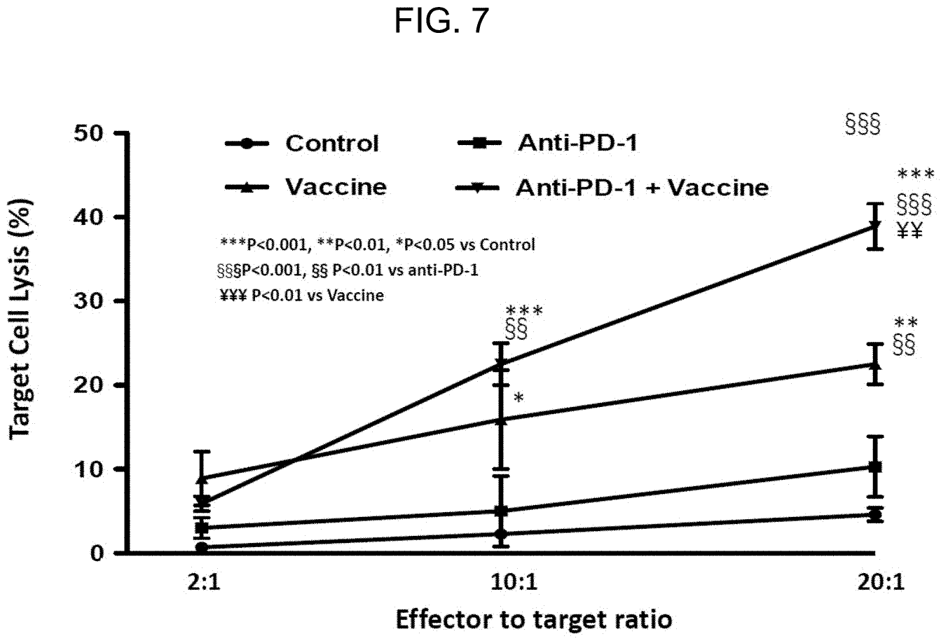

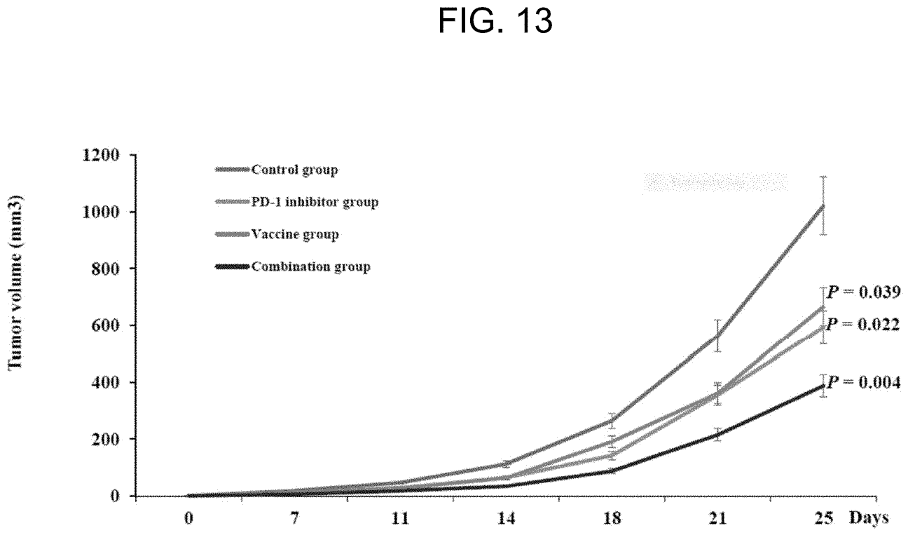

In any of the methods or compositions disclosed herein, administration of an effective amount of a composition comprising a bacteriophage expressing a fragment of human ASPH and an effective amount of an immune checkpoint protein inhibitor may have a synergistic effect. As used herein, “synergy” or “synergistic effect” with regard to an effect produced by two or more individual components refers to a phenomenon in which the total effect produced by these components, when utilized in combination, is greater than the sum of the individual effects of each component acting alone. For example, the combination of a composition comprising a bacteriophage expressing a fragment of human ASPH and anti-PD-1 antibody checkpoint protein inhibitor showed a synergistic effect over either the bacteriophage composition alone or the anti-PD-1 antibody alone, as demonstrated in FIGS. 5-7 and FIG. 13.

In some embodiments, the composition comprising a bacteriophage expressing a fragment of human ASPH is a nanoparticle vaccine.

In any of the methods or compositions disclosed herein, the bacteriophage may be bacteriophage lambda. In some embodiments, the bacteriophage expresses an immunogenic fragment of human ASPH. In some embodiments, a bacteriophage (e.g., bacteriophage lambda) comprises a fusion protein comprising a portion of the ASPH protein fused to bacteriophage lambda head decoration protein D (gpD) or a portion of gpD. In some embodiments, a portion of the ASPH protein is fused at the C-terminus of gpD or a portion of gpD. In some embodiments, the portion of the gpD in the fusion protein comprises or consists of SEQ ID NO: 2.

(SEQ ID NO: 2)
HMTSKETFTHYQPQGNSDPAHTATAPGGLSAKAPAMTPLMLDTSSRKLVA
WDGTTDGAAVGILAVAADQTSTTLTFYKSGTFRYEDVLWPEAASDETKKR
TAFAGTAISIV

In some embodiments, the fusion protein comprises a linker sequence between the gpD amino acid sequence and the ASPH amino acid sequence. In some embodiments, a linker sequence comprises or consists of GGSGPVGPGGSGAS (SEQ ID NO: 3).

In some embodiments, a bacteriophage comprises a fusion protein comprising a gpD or a portion of gpD and an antigenic fragment of at least 9 amino acids, at least 15 amino acids, at least 20 amino acids, at least 25 amino acids, at least 30 amino acids, at least 35 amino acids, at least 40 amino acids, at least 45 amino acids, at least 50 amino acids, at least 75 amino acids or at least 100 amino acids from any one of SEQ ID NO: 4, SEQ ID NO: 5, SEQ ID NO: 6 and SEQ ID NO:7 (Table 1). In some embodiments, a bacteriophage comprises a fusion protein comprising a gpD or a portion of gpD and an ASPH fragment consisting of the amino acid sequence of SEQ ID NO: 4, SEQ ID NO: 5, SEQ ID NO: 6 or SEQ ID NO:7. In some embodiments, a fusion protein comprising a portion of the ASPH protein fused to gpD does not comprise any sequence from the ASPH amino acid sequence having homology to human Junctin protein and/or human Humbug protein.

TABLE 1
Illustrative human ASPH fragment sequences

Fragment I
(SEQ ID NO: 4)
STSEPAVPPEEAEPHTEPEEQVPVEAEPQNIEDEAKEQIQSLLHEMVHAE
HVEGEDLQQEDGPTGEPQQEDDEFLMATDVDDRFETLEPEVSHEETEHSY
HVEETVSQDCNQDMEEMMSEQENPDSSEPVVEDERLHHDTDDVTYQVYEE
QAVYEPLENEGIEITEVTAPPEDNPVEDSQVIVEEVSIFPVEEQQEVPP
Fragment Ia
(SEQ ID NO: 5)
DRAMAQRKNAKSSGNSSSSGSGSGSTSAGSSSPGARRETKHGGHKNGRKG
GLSGTSFFTWFMVIALLGVWTSVAVVWFDLVDYEEVLGKLGIYDADGDGD
FDVDDAKVLLGLKERSTSEPAVPPEEAEPHTEPEEQVPVEAEPQNIEDEA
KEQIQSLLHEMVHAEHVEGEDLQQEDGPTGEPQQEDDEFLMATDVDDRFE
TLEPEVSHEETEHSYHVEETVSQDCNQDMEEMMSEQENPDSSEPVVEDER
LHHDTDDVTYQVYEEQAVYEPLENEGIEITEVTAPPEDNPVEDSQVIVEE
VSIFPVEEQQEVPP
Fragment II
(SEQ ID NO: 6)
LDAAEKLRKRGKIEEAVNAFKELVRKYPQSPRARYGKAQCEDDLAEKRRS
NEVLRGAIETYQEVASLPDVPADLLKLSLKRRSDRQQFLGHMRGSLLTLQ
RLVQLFPNDTSLKNDLGVGYLLIGDNDNAKKVYEEVLSVTPNDGFAKVHY
GFILKAQNKIAESIPYLKEGIESGDP
Fragment III
(SEQ ID NO: 7)
GTDDGRFYFHLGDAMQRVGNKEAYKWYELGHKRGHFASVWQRSLYNVNGL
KAQPWWTPKETGYTELVKSLERNWKLIRDEGLAVMDKAKGLFLPEDENLR
EKGDWSQFTLWQQGRRNENACKGAPKTCTLLEKFPETTGCRRGQIKYSIM
HPGTHVWPHTGPTNCRLRMHLGLVIPKEGCKIRCANETRTWEEGKVLIFD
DSFEHEVWQDASSFRLIFIVDVWHPELTPQQRRSLPAI

In some embodiments the bacteriophage lambda expresses amino acids 113-311 from the N-terminal region of ASPH fused at the C-terminus of gpD or a portion of gpD. In some embodiments, the bacteriophage lambda expresses a fusion protein comprising or consisting of the amino acid sequence of SEQ ID NO: 1. In some embodiments, the bacteriophage lambda expresses a fusion protein comprising an amino acid sequence having at least 92%, at least 93%, at least 94%, at least 95%, at least 96%, at least 97%, at least 98% or at least 99% sequence identity to the amino acid sequence of SEQ ID NO: 1.

In any of the methods or compositions disclosed herein, the recombinant bacteriophage expressing a fragment of human ASPH is provided as a pharmaceutical composition. In some embodiments, a pharmaceutical composition is a nanoparticle vaccine. In some embodiments, the recombinant bacteriophages are inactivated by ultraviolet radiation to produce nanoparticles. The nanoparticles are the drug substances. Examples of methods of producing nanoparticle vaccines comprising recombinant bacteriophages are provided in U.S. Pat. No. 9,744,223, US 2017/0072034 and WO 2020/081996. In some embodiments, a nanoparticle vaccine does not comprise an adjuvant (e.g., does not comprise an exogenous adjuvant). Bacteriophage is innately immunogenic and usually requires no exogenous adjuvant.

As used herein, “immune checkpoint” refers to co-stimulatory and inhibitory signals that regulate the amplitude and quality of T-cell receptor recognition of an antigen. In certain embodiments, the immune checkpoint is an inhibitory signal. In some embodiments, the inhibitory signal is the interaction between Programmed Death-1 (PD-1) and Programmed Death Ligand-1 (PD-L1).

As used herein, “immune checkpoint protein inhibitor” refers to a molecule that reduces, inhibits, interferes with or modulates one or more immune checkpoint proteins. In some embodiments, the immune checkpoint protein inhibitor prevents inhibitory signals associated with the immune checkpoint. In some embodiments, the immune checkpoint protein inhibitor is an antibody, or fragment thereof, that disrupts inhibitory signaling associated with the immune checkpoint. In some embodiments, the immune checkpoint protein inhibitor is a PD-1, PD-L1, PD-L2, CTLA4, A2AR, B7-H3, BTLA, IDO, KIR, LAG3, TIM-3, or VISTA inhibitor. In some embodiments, the immune checkpoint protein inhibitor is a small molecule that disrupts inhibitory signaling. In some embodiments, the immune checkpoint protein inhibitor is an antibody, fragment thereof, or antibody mimic, that prevents the interaction between immune checkpoint proteins, e.g., an antibody, or fragment thereof, that prevents or disrupts the interaction between PD-1 and PD-L1. The immune checkpoint protein inhibitor may also be in the form of the soluble form of the molecules (or mutated versions thereof) themselves, e.g., a soluble PD-L1 or PD-L1 fusion.

In any of the methods or compositions disclosed herein, the immune checkpoint protein may be PD-1 (e.g., human PD-1). The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773.

In any of the methods or compositions disclosed herein, the immune checkpoint protein may be PD-L1 (e.g., human PD-L1). “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

In any of the methods and compositions disclosed herein, the immune checkpoint protein may be a component of the PD-1/PD-L1 signaling pathway. Accordingly, certain embodiments provide methods for immunotherapy of a subject afflicted with cancer, wherein the methods comprise administering to the subject a therapeutically effective amount of an antibody or an antigen-binding portion thereof that disrupts the interaction between the human PD-1 receptor and its ligand, human PD-L1. Antibodies known in the art which bind to PD-1 and disrupt the interaction between the PD-1 and its ligand, PD-L1, and stimulate an anti-tumor immune response, are suitable for use in the methods disclosed herein. In some embodiments, the antibody or antigen-binding portion thereof binds specifically to or targets PD-1. For example, antibodies that target PD-1 include, e.g., nivolumab (BMS-936558, Bristol-Myers Squibb) and pembrolizumab (lambrolizumab, MK03475, Merck). Other suitable antibodies for use in the methods disclosed herein are anti-PD-1 antibodies disclosed in U.S. Pat. No. 8,008,449, herein incorporated by reference in its entirety. In certain embodiments, the antibody or antigen-binding portion thereof binds specifically to or targets PD-L1 and inhibits its interaction with PD-1. Antibodies known in the art which bind to PD-L1 and disrupt the interaction between the PD-1 and PD-L1, and stimulates an anti-tumor immune response, are suitable for use in the methods disclosed herein. For example, antibodies that target PD-L1 include BMS-936559 (also known as MDX 1105, Bristol-Myers Squibb), atezolizumab (Genentech) and avelumab (MSB0010718C). Other suitable antibodies that target PD-L1 are disclosed in U.S. Pat. No. 7,943,743, herein incorporated by reference in its entirety. Any antibody that binds specifically to PD-1 or PD-L1, disrupts the PD-1/PD-L1 interaction, and stimulates an anti-tumor immune response, is suitable for use in the methods and compositions disclosed herein.

Suitable subjects (e.g., patients) that may be treated by the methods and compositions disclosed herein include laboratory animals (such as mouse, rat, rabbit, or guinea pig), farm animals, and domestic animals or pets (such as a cat or dog). In some embodiments, a subject is a human or a non-human primate. In some embodiments, a subject is a human who has an ASPH-expressing cancer, has been diagnosed with an ASPH-expressing cancer, or is at risk of having an ASPH expressing cancer.

In some embodiments, a subject may be refractory to treatment with a monotherapy comprising administration of an immune checkpoint protein inhibitor. In some embodiments, a subject may have evidence of progressive disease while on a monotherapy comprising administration of an immune checkpoint protein inhibitor. In some embodiments, the monotherapy is a PD-L1 inhibitor or a PD-1 inhibitor. In some embodiments, the monotherapy is pembrolizumab or nivolumab. In some embodiments, a subject may be experiencing a relapse of cancer. In some embodiments, a combination therapy disclosed herein may be administered to a subject prior to surgery to resect a tumor or a portion of a tumor from the subject.

In some embodiments, the composition comprising a bacteriophage expressing a fragment of human ASPH is administered intravenously or intradermally. In some embodiments, the composition comprising a bacteriophage expressing a fragment of human ASPH is administered using a hollow microstructured transdermal system (hMTS) device (3M®).

In some embodiments, the composition comprising a bacteriophage expressing a fragment of human ASPH (e.g., a nanoparticle vaccine comprising bacteriophage lambda expressing a fusion protein comprising or consisting of the amino acid sequence of SEQ ID NO: 1) is administered at a dose from about 2×10¹⁰ particles up to about 3×10¹¹ particles. In some embodiments, the nanoparticle vaccine is administered at a dose of about 2×10¹⁰ particles, about 1×10¹¹ particles or about 3×10¹¹ particles. In some embodiments, the nanoparticle vaccine is administered to a subject at a dose of about 1×10¹¹ particles. In some embodiments, the nanoparticle vaccine is administered at a dose of about 1×10¹¹ particles in a 1 ml intradermal injection. In some embodiments, the nanoparticle vaccine is administered every 3 weeks (±3 days) for 4 doses then every 6 weeks (±3 days) for 6 additional doses, thereafter every 12 weeks (±3 days). In some embodiments, the nanoparticle vaccine is administered for up to 24 months.

In some embodiments, the immune checkpoint protein inhibitor is administered intravenously.

In some embodiments, the immune checkpoint protein inhibitor is pembrolizumab and is administered at a dose of about 200 mg. In some embodiments, the immune checkpoint protein inhibitor (e.g., pembrolizumab) is administered at a dose of about 200 mg as an intravenous infusion over about 30 minutes every 3 weeks (±3 days). In some embodiments, the immune checkpoint protein inhibitor (e.g., pembrolizumab) is administered for up to 24 months.

In some embodiments, the composition comprising a bacteriophage expressing a fragment of human ASPH (e.g., a nanoparticle vaccine comprising bacteriophage lambda expressing a fusion protein comprising or consisting of the amino acid sequence of SEQ ID NO: 1) is administered after the immune checkpoint protein inhibitor. In some embodiments, the composition comprising a bacteriophage expressing a fragment of human ASPH (e.g., a nanoparticle vaccine comprising bacteriophage lambda expressing a fusion protein comprising or consisting of the amino acid sequence of SEQ ID NO: 1) is administered before the immune checkpoint protein inhibitor.

In some embodiments, the disclosure provides a method for treating or ameliorating cancer or a symptom of cancer in a subject, the method comprising administering to the subject an effective amount of a composition comprising a bacteriophage expressing a fragment of human ASPH and an effective amount of an immune checkpoint protein inhibitor, wherein the composition comprising the bacteriophage comprises bacteriophage lambda expressing a fusion protein comprising or consisting of the amino acid sequence of SEQ ID NO: 1; wherein the composition comprising the bacteriophage is administered at a dose of about 1×10¹¹ particles in a 1 ml intradermal injection (e.g., using a micro-needle device) every 3 weeks (±3 days) for 4 doses then every 6 weeks (±3 days) for 6 additional doses, thereafter every 12 weeks (±3 days), wherein the immune checkpoint protein inhibitor is pembrolizumab, and wherein the pembrolizumab is administered at a dose of about 200 mg as an intravenous infusion over about 30 minutes every 3 weeks. In some embodiments, the nanoparticle vaccine and the immune checkpoint protein inhibitor (e.g., pembrolizumab) are administered for up to 24 months. In some embodiments, the nanoparticle vaccine is administered approximately 1 hour after intravenous infusion of pembrolizumab for the first dose. In some embodiments, subsequent doses of nanoparticle vaccine and pembrolizumab can be administered in any order. In some embodiments, the cancer is ASPH-positive squamous cell cancer of the head and neck (SCCHN). In some embodiments, the cancer is ASPH-positive locally advanced unresectable or metastatic/recurrent SCCHN.

In some embodiments, the disclosure also provides a composition comprising: a pharmaceutical composition comprising a bacteriophage expressing a fragment of human ASPH; and a pharmaceutical composition comprising an immune checkpoint protein inhibitor. In some embodiments, said pharmaceutical compositions are in separate containers. In some embodiments, the bacteriophage is a bacteriophage lambda expressing a fusion protein comprising or consisting of the amino acid sequence of SEQ ID NO: 1. In some embodiments, the pharmaceutical composition comprising the bacteriophage is formulated for administration at a dose of about 1×10¹¹ particles in a 1 ml intradermal injection (e.g., using a micro-needle device). In some embodiments, the immune checkpoint protein inhibitor is pembrolizumab. In some embodiments, the pembrolizumab is formulated for administration at a dose of about 200 mg as an intravenous infusion.

### EXAMPLES

### Example 1A: Effects Of Combination Therapy On Hepatocellular Carcinoma (HCC)

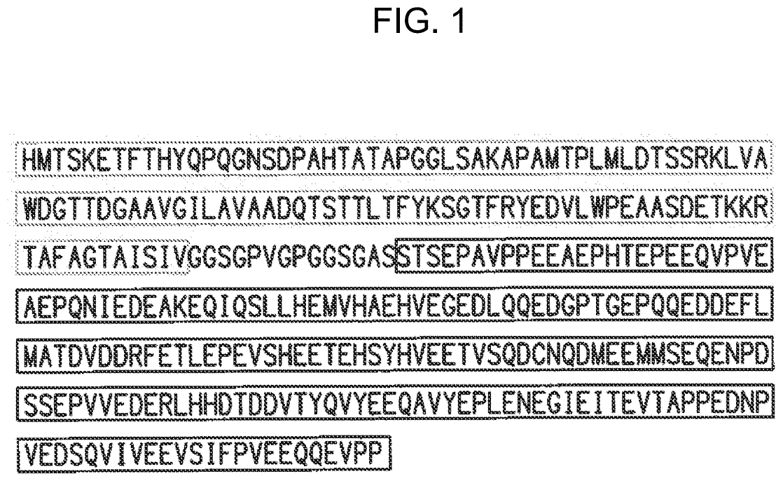

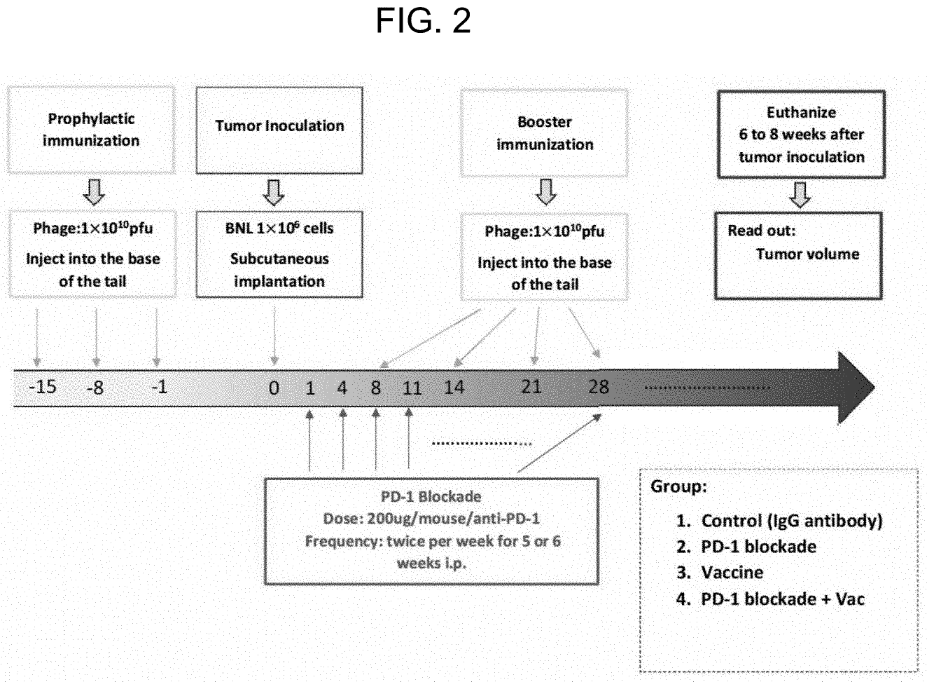

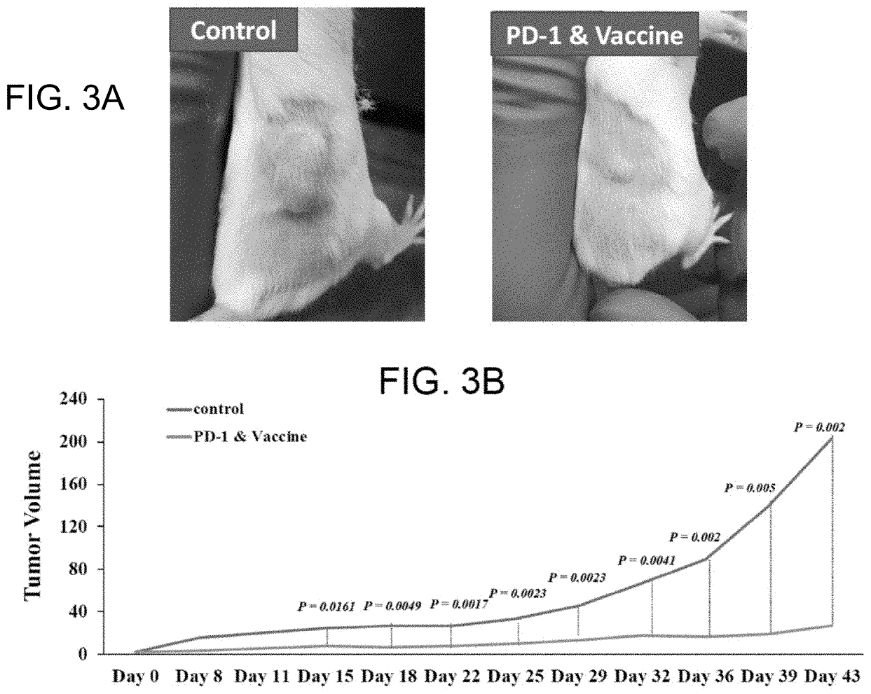

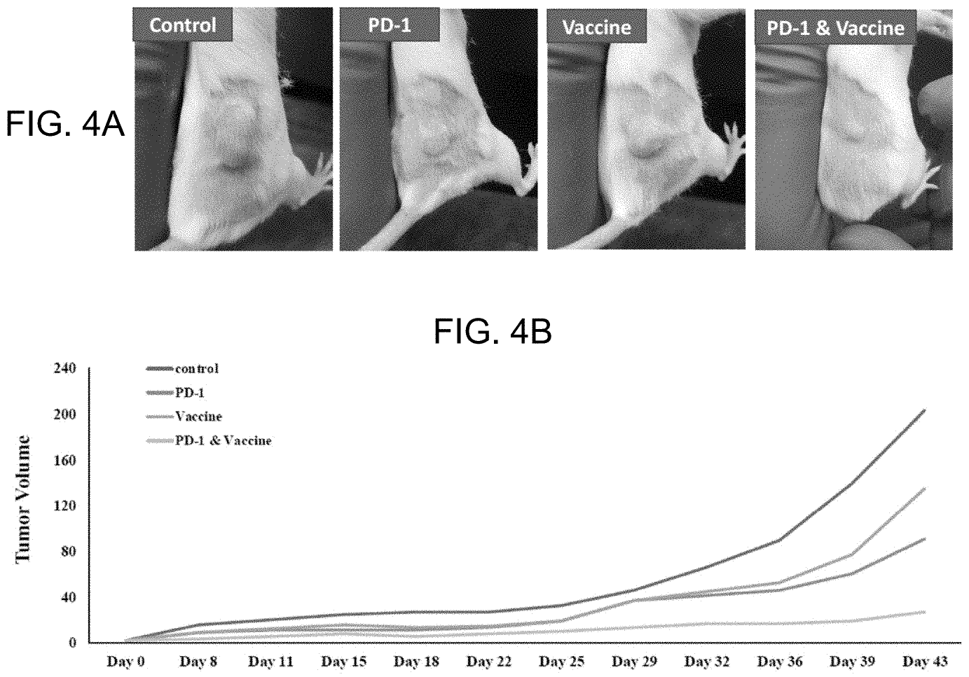

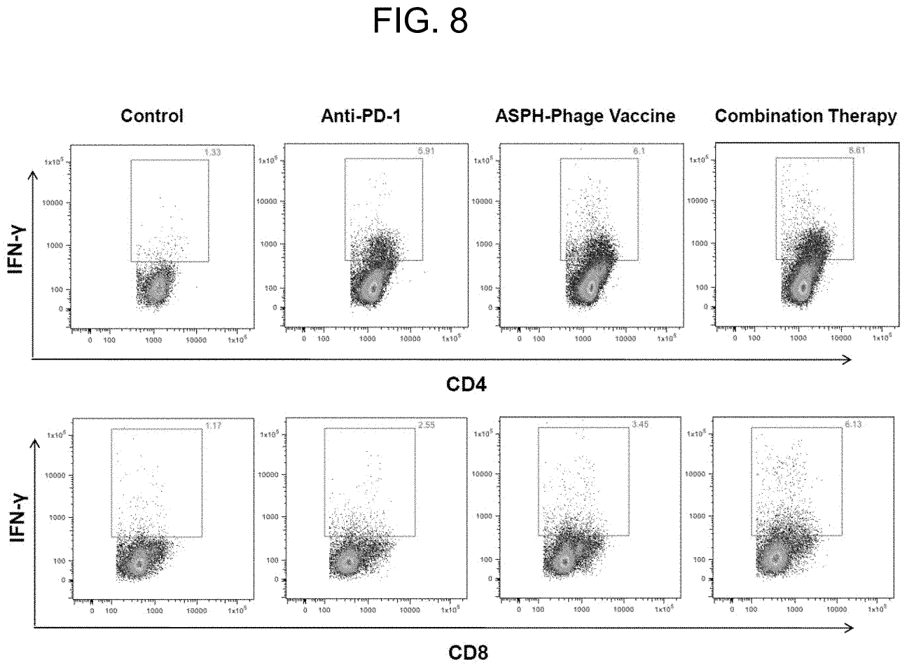

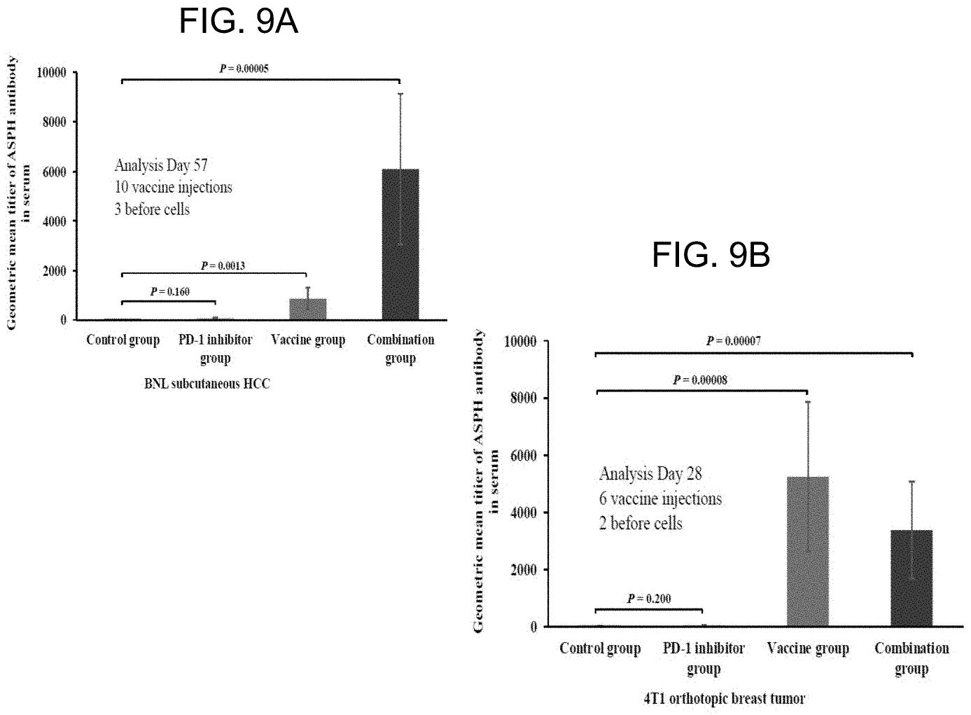

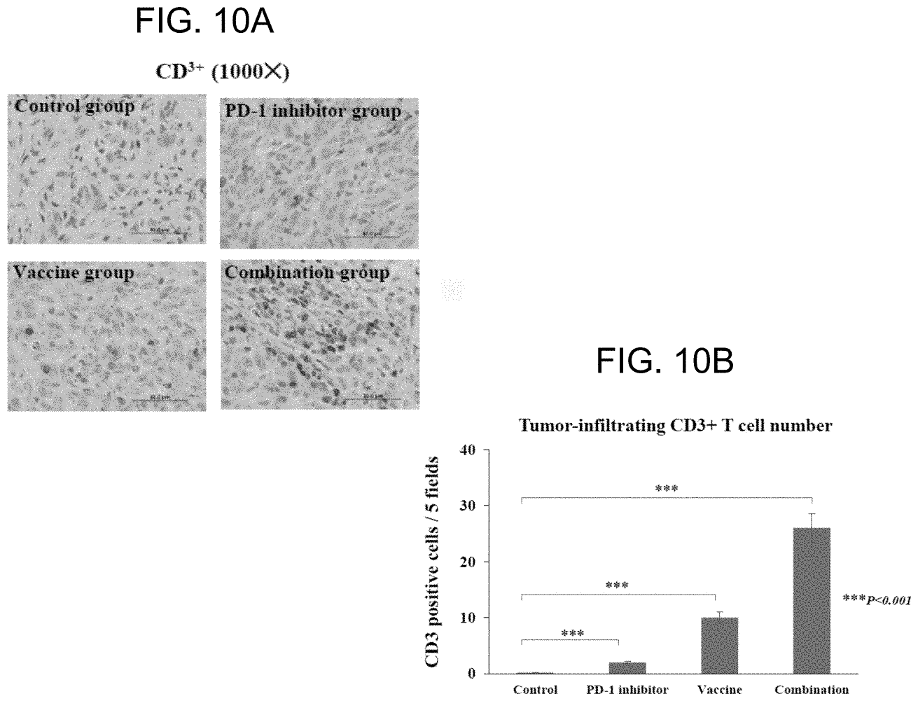

The effects of treatment with (1) a composition comprising a bacteriophage expressing a fragment of human ASPH, (2) an anti-PD-1 antibody or (3) a combination of a composition comprising a bacteriophage expressing a fragment of human ASPH on BNL cells were evaluated in vitro and in an animal model. “BNL” refers to the BNLT3 cell line, a BALB/c-derived hepatocellular carcinoma cell line that produces solid tumors when administered subcutaneously and metastatic tumors when injected into the spleen or peritoneum. Experimental protocols and results from these experiments are shown in FIG. 2-FIG. 8, FIG. 9A and FIG. 10. Throughout these figures, “vaccine” refers to treatment with a bacteriophage lambda nanoparticle vaccine, wherein the bacteriophage lambda expresses a protein having the amino acid sequence of SEQ ID NO: 1 (see FIG. 1). Throughout these figures, “PD-1”, “PD-1 blockade” and “anti-PD-1” refers to treatment with the InVivoMAb anti-mouse PD-1 (CD279) monoclonal antibody (Clone: RMP1-14).

### Example 1B: Effects Of Combination Therapy On Breast Cancer

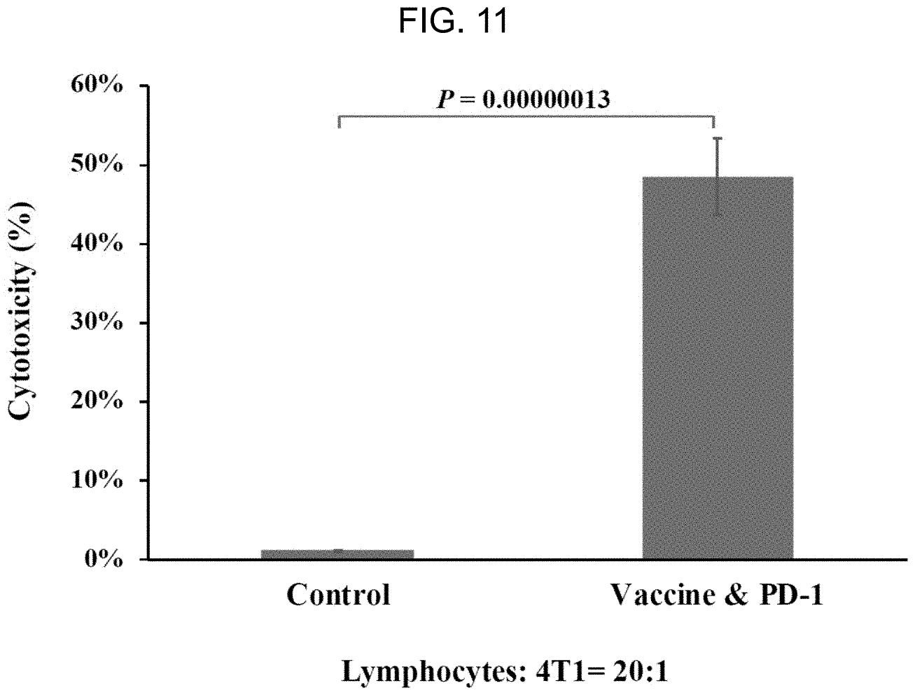

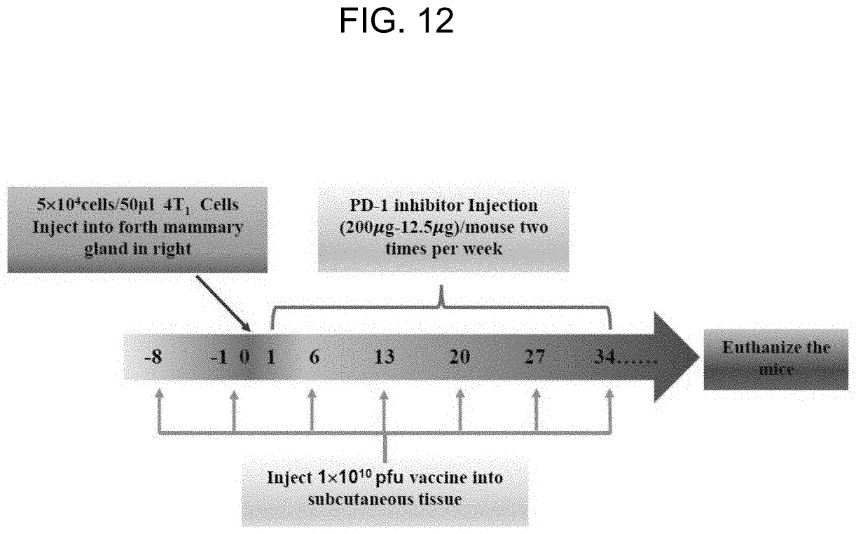

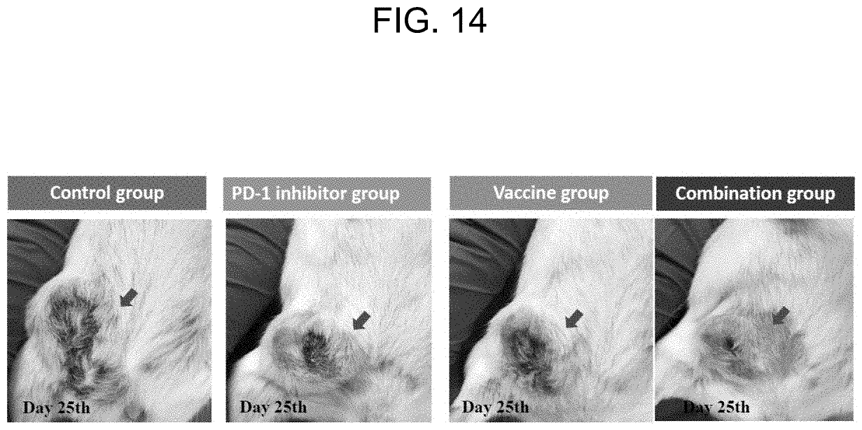

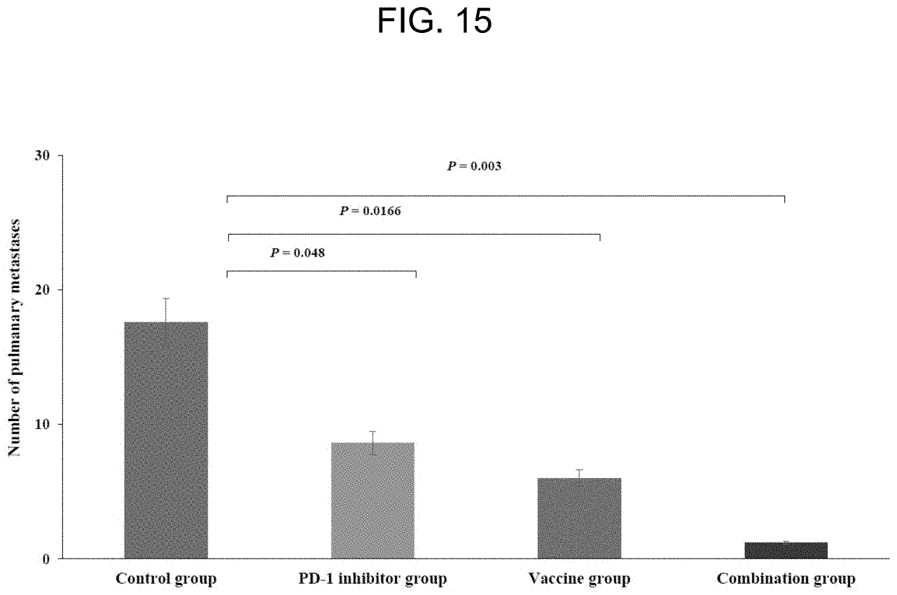

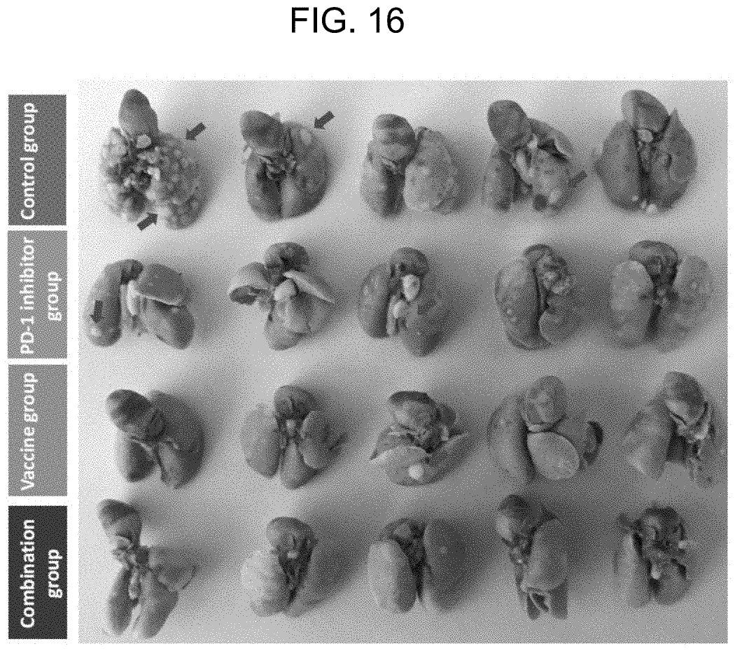

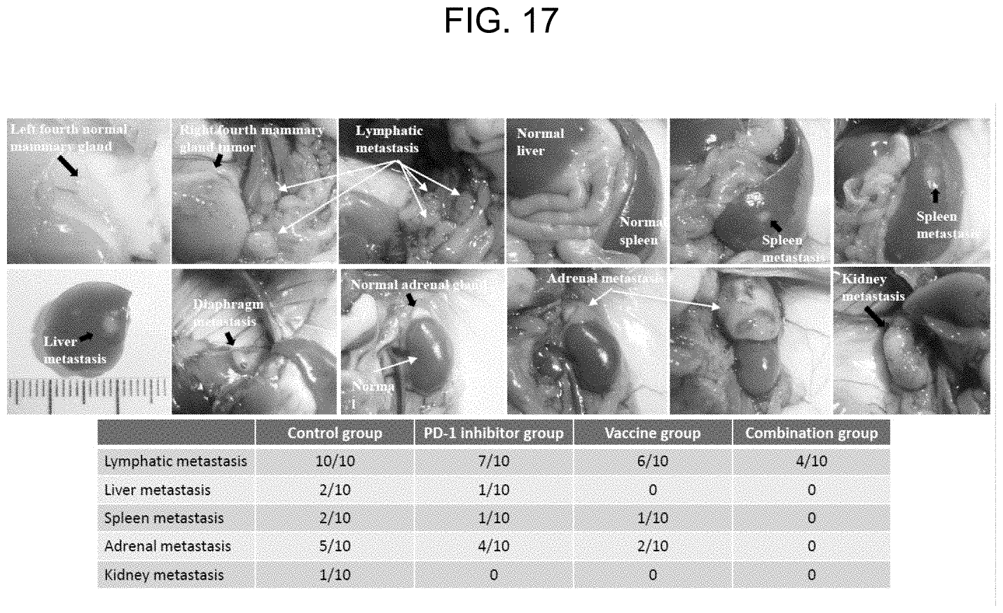

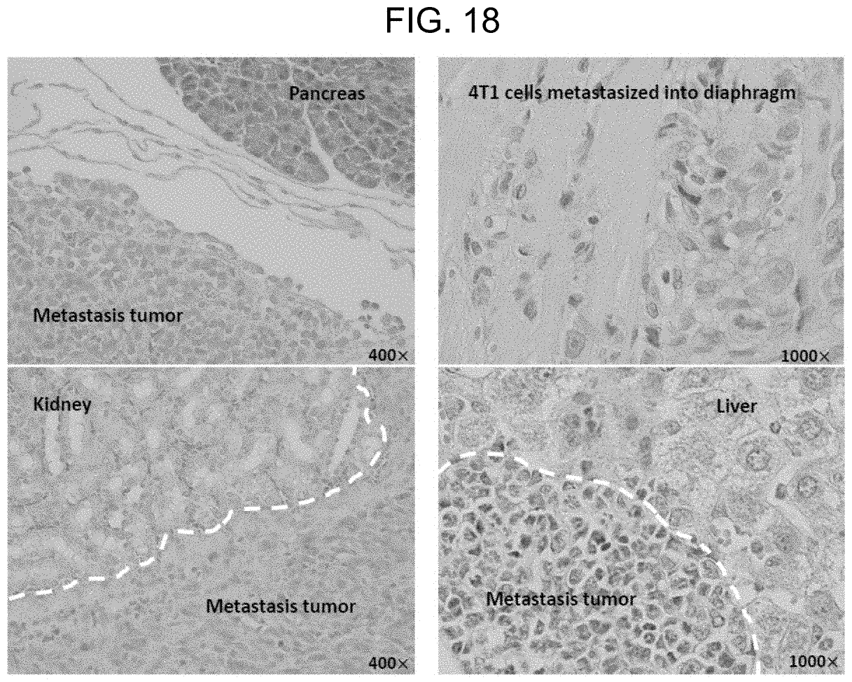

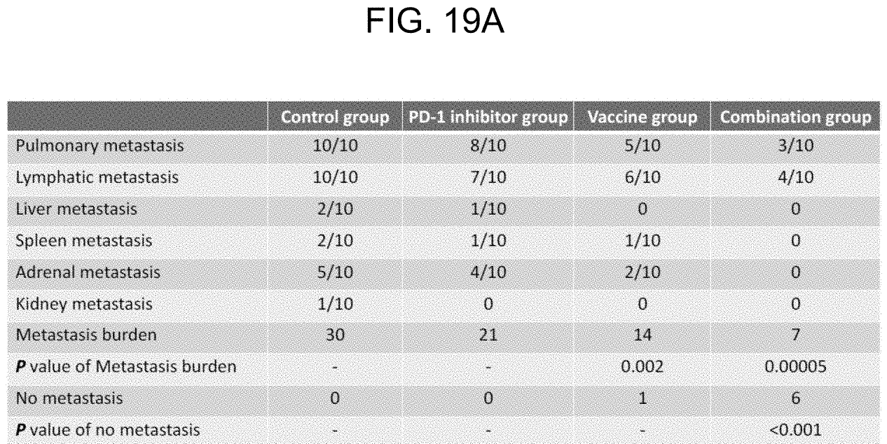

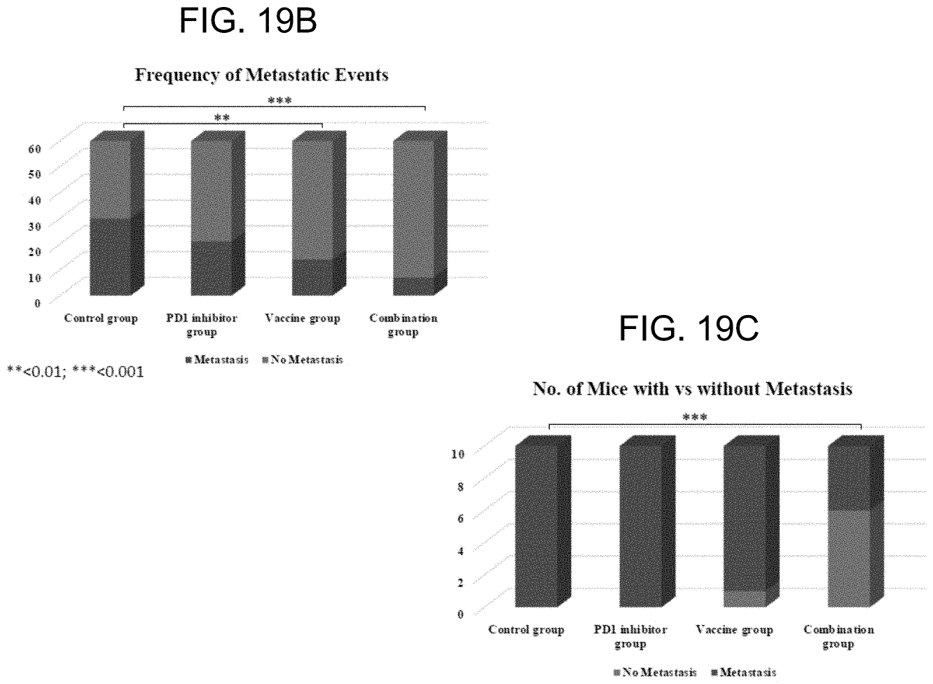

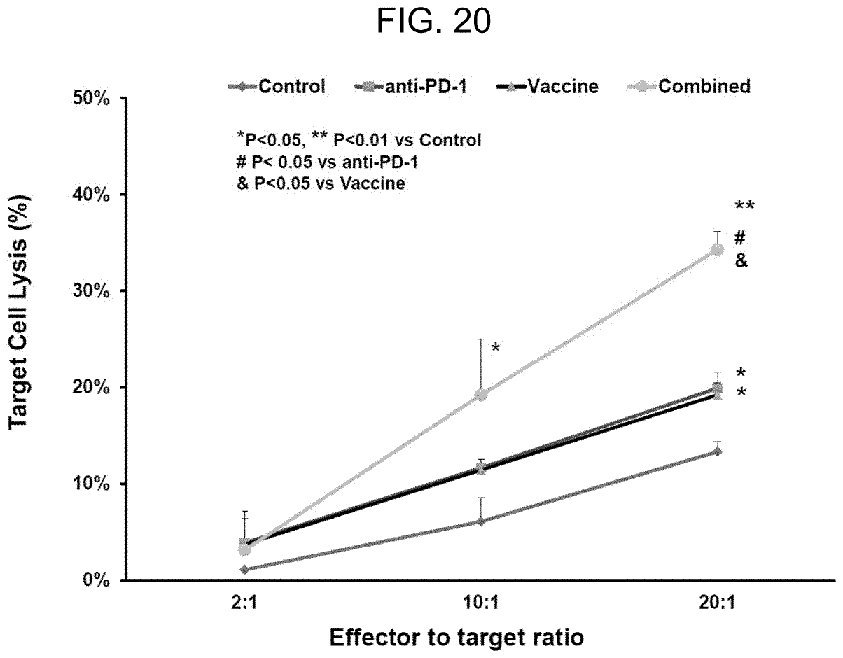

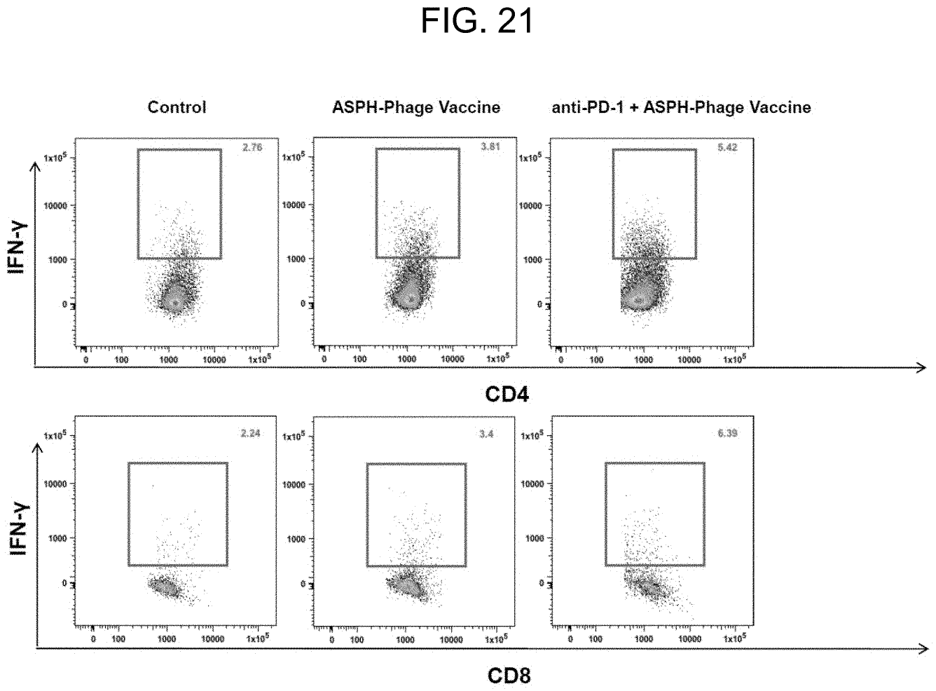

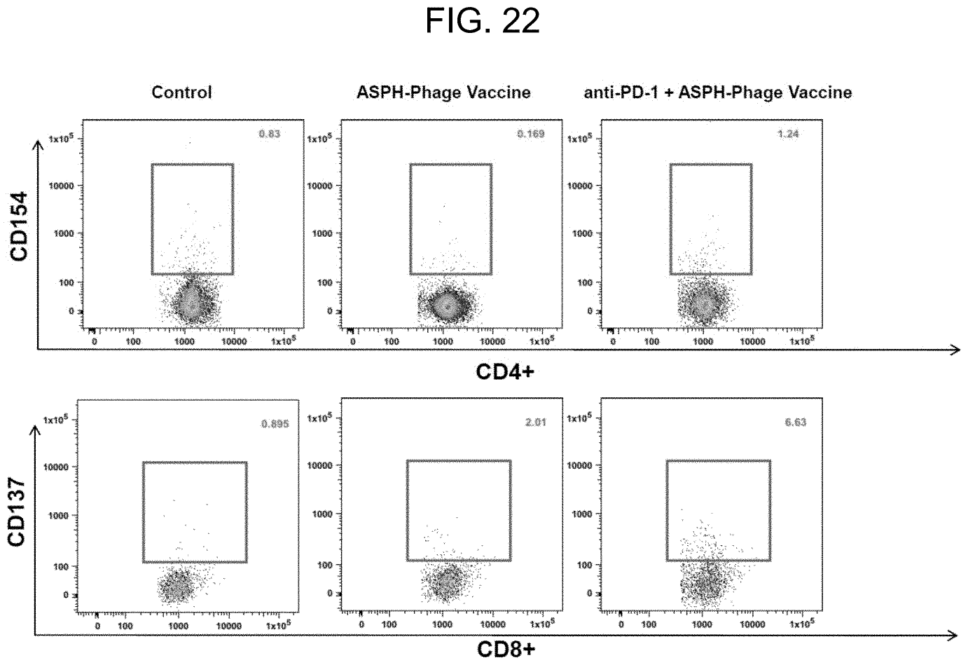

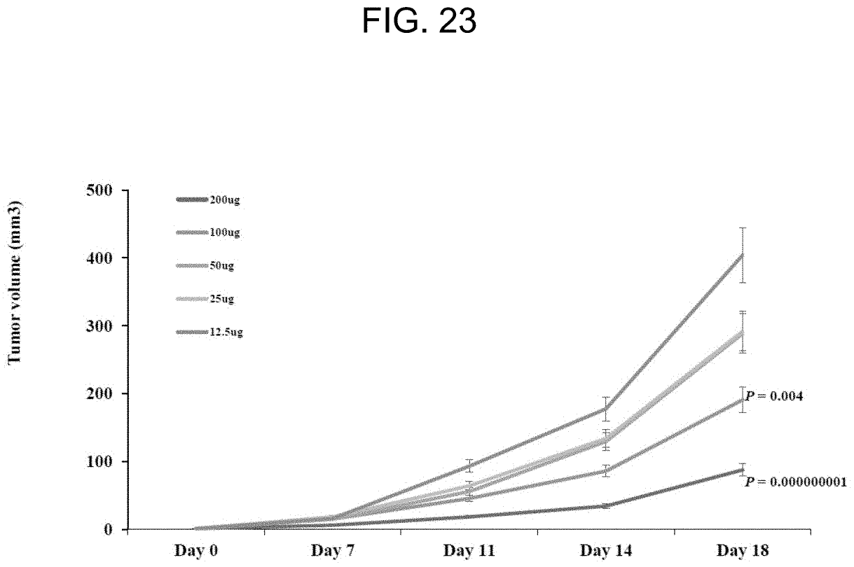

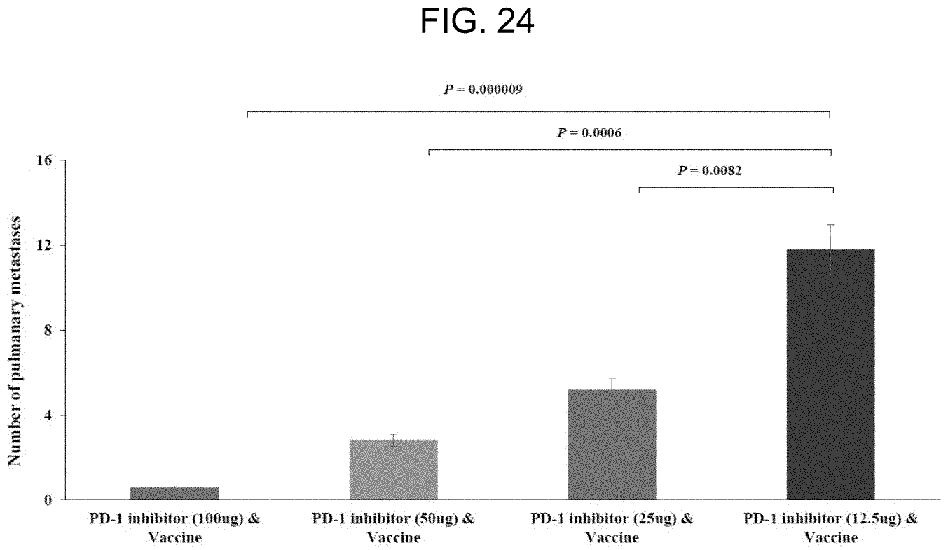

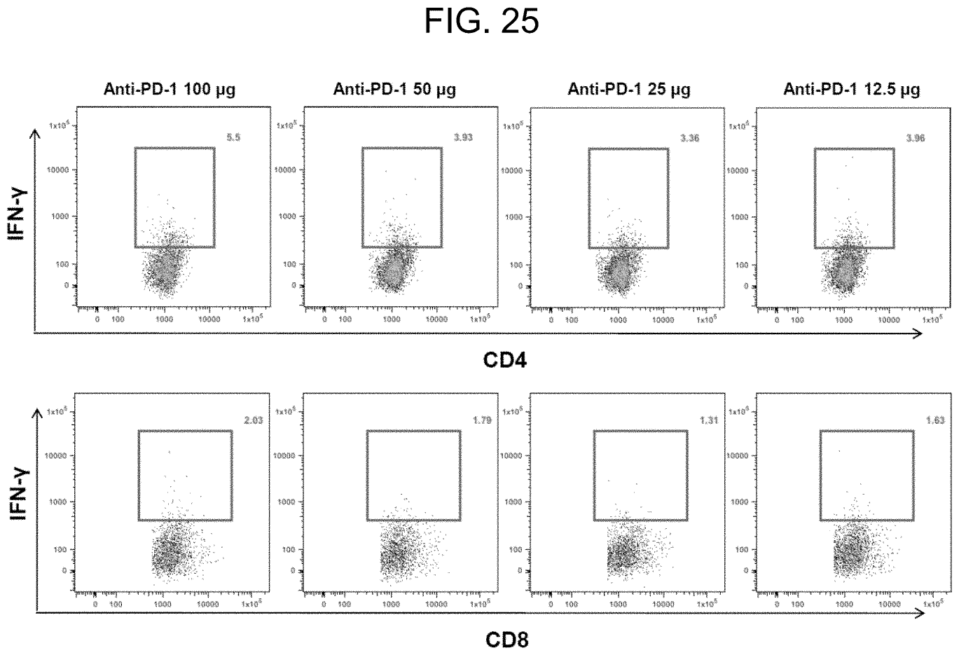

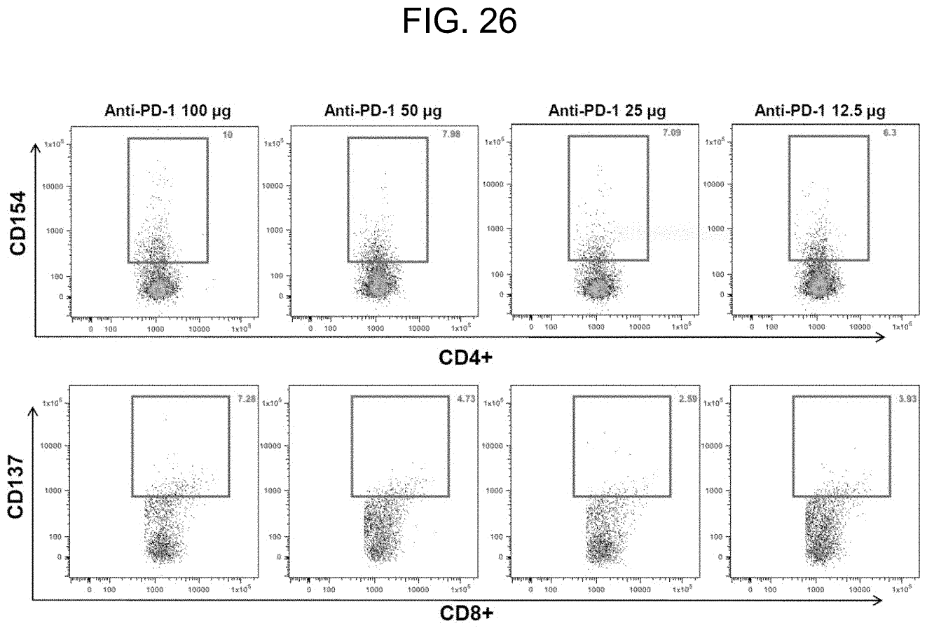

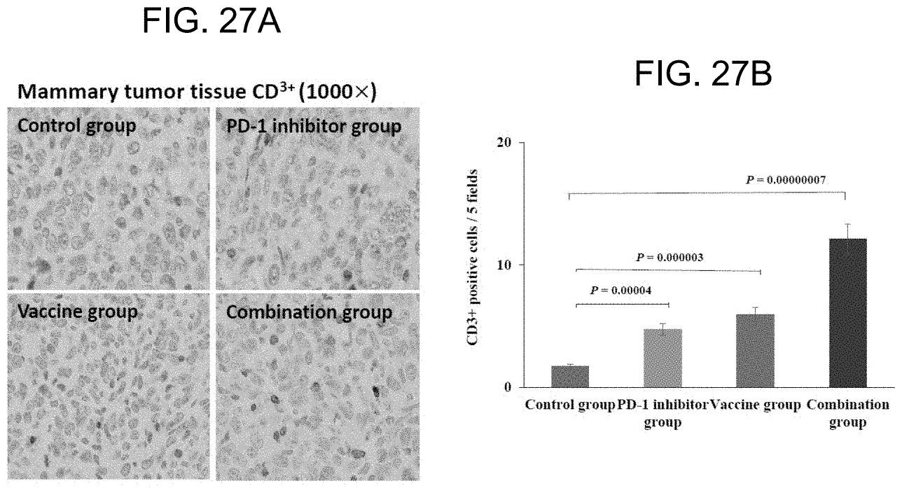

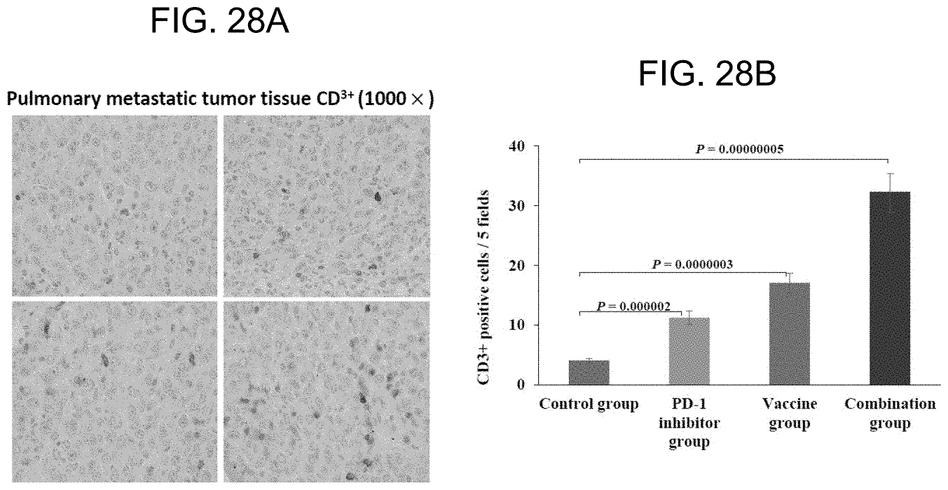

The effects of treatment with (1) a composition comprising a bacteriophage expressing a fragment of human ASPH, (2) an anti-PD-1 antibody or (3) a combination of a composition comprising a bacteriophage expressing a fragment of human ASPH on an orthotopic 4T1 murine breast cancer model were evaluated in vitro and in an animal model. Experimental protocols and results from these experiments are shown in FIG. 9B and FIG. 11-FIG. 29D. Throughout these figures, “vaccine” refers to treatment with a bacteriophage lambda nanoparticle vaccine, wherein the bacteriophage lambda expresses a protein having the amino acid sequence of SEQ ID NO: 1 (FIG. 1). Throughout these figures, “PD-1”, “PD-1 blockade” and “anti-PD-1” refers to treatment with the InVivoMAb anti-mouse PD-1 (CD279) monoclonal antibody (Clone: RMP1-14).

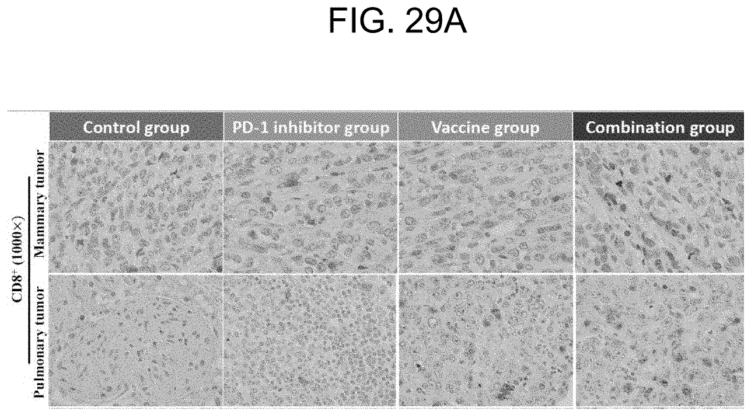

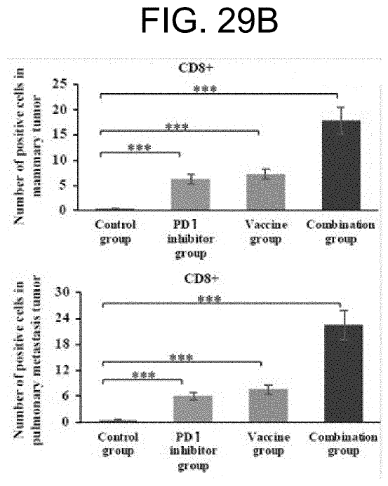

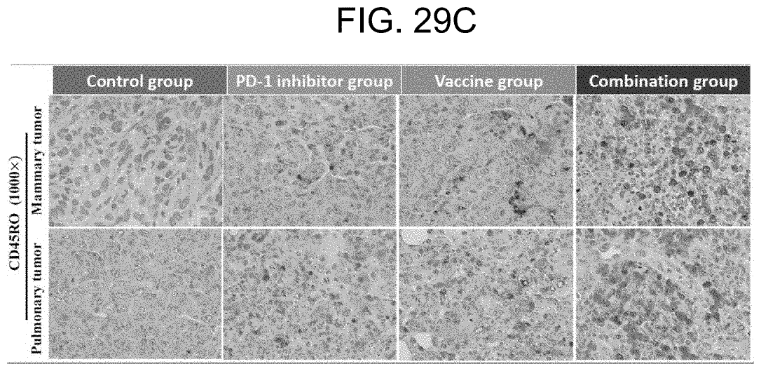

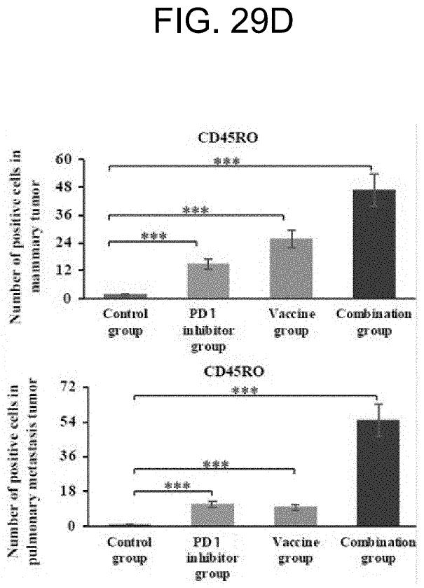

FIG. 29A-FIG. 29D show accumulation of CD8+ T cells in the combination treatment group of tumors, both in the primary tumor as well as the metastatic tumors compared to the other three groups. These cytotoxic T cells bear the CD45RO cell surface marker, indicating that they are also activated CD8+ memory T cells activated by bacteriophage lambda vaccination and may persist to continue to attack ASPH-positive tumor cells. The implication is that the bacteriophage lambda is behaving like a true vaccine and may provide long-term anti-tumor activity and protection. The data also suggest that the immune checkpoint protein inhibitor releases the antigen-specific CD8+ cytotoxic T cells to produce this potent immune attack and prevents metastasis in 60% of the vaccinated mice, which previously has not been observed.

### Example 1C: Conclusions

A single well-defined and pure tumor-associated antigen was sufficient to generate robust anti-tumor responses to ASPH in HCC and triple negative breast cancer. The bacteriophage lambda vaccine activated ASPH-specific humoral and cellular immune responses that produced potent anti-tumor effects in vivo. The bacteriophage lambda vaccine was equal to or superior in generating antigen specific cellular and humoral immune responses as well as therapeutic anti-tumor effects compared to the anti-PD-1 checkpoint inhibitor antibody.

The dose response curve of the anti-PD-1 antibody effects showed that a 50% reduction in amount still produced inhibition of antigen specific cellular and humoral immune responses as well as reducing tumor spread but it is substantially less than the full dose (200 mg×2 per week).

The bacteriophage lambda vaccine activated ASPH-specific cellular and humoral immune responses, and the anti-PD-1 antibody substantially amplified this response to achieve greater therapeutic activity. The combination of anti-PD-1 antibody and bacteriophage lambda vaccination promoted tumor infiltrating CD3+ T cells (TILS) in breast and liver cancer models. For the first time, antigen-specific TILS were found in the metastatic lesions with the highest numbers associated with combination therapy. There was a striking reduction in the metastatic burden in the combination therapy treated mice compared to the other 3 groups. Analysis of the total metastatic burden revealed a substantial protective effect of the combination therapy with 60% of the mice so treated having no detectable metastases compared to the other 3 groups.

These results suggest that the combination treatments disclosed herein will be an important advance in cancer immunotherapy for patients with “difficult to treat” solid tumors.

### Example 2: Phase 1/2 Clinical Trial Of Administration Of A Composition Comprising A Bacteriophage Expressing A Fragment Of Human ASPH In Addition To Pembrolizumab To Treat Head-And-Neck Cancer

A Phase 1/2, open-label, multi-center trial to evaluate the safety, immunogenicity and preliminary clinical efficacy of SNS-301 delivered intradermally in addition to pembrolizumab in patients with ASPH+ locally advanced unresectable or metastatic/recurrent squamous cell cancer of the head and neck (SCCHN) is performed. SNS-301 is a nanoparticle vaccine drug substance, which is a recombinant bacteriophage lambda construct that is engineered to display a fusion protein of phage gpD and a portion of the human ASPH protein sequence. The HAAH-1λ (SNS-301) construct contains 199 amino acids from the N-terminal region (amino acids 113-311) of the molecule. The recombinant bacteriophage lambda in SNS-301 expresses on its surface a fusion protein comprising SEQ ID NO: 1.

The trial population consists of patients with ASPH+ locally advanced unresectable or metastatic/recurrent SCCHN who are currently receiving a PD-L1 inhibitor therapy or a PD-1 inhibitor therapy (e.g., pembrolizumab or nivolumab). Patients must have a best response of stable disease (SD) or first evidence of progressive disease (PD) after a minimum of 12 weeks of pembrolizumab or nivolumab therapy. Patients may or may not have received platinum-based therapy with evidence of disease progression prior to initiation of pembrolizumab or nivolumab. Patients receiving first-line pembrolizumab monotherapy prior to this study must be PD-L1 positive. Patients receiving non-pembrolizumab therapy will be switched over to pembrolizumab at the time of entering this study.

Approximately 30 patients will be enrolled in the trial.

This is a two-stage clinical trial with the primary efficacy endpoint of objective response per iRECIST (Immune response evaluation criteria in solid tumors) at 12 weeks. Patients enrolled in the first stage will need to be deemed evaluable at 12 weeks, meeting the definition for the efficacy evaluable analysis set; at least one dose of treatment and the Week 12 efficacy assessment or progression prior to Week 12. Approximately 15 patients will be enrolled in the first stage and evaluated for objective response (futility assessment) at 12 weeks. All patients will participate in the overall efficacy analysis. If warranted, based on response, an additional 15 patients will be enrolled in the second stage.

After consenting to participate in this clinical trial, participants will be screened for enrollment. A positive ASPH tissue sample is required for entry onto the study with either a fresh biopsy or archival tissue from a previous biopsy. Ideally, a pre-treatment tissue sample obtained after initiation of ongoing PD-L1 inhibitor therapy or a PD-1 inhibitor therapy and first dose of SNS-301 and pembrolizumab on this current clinical trial will be collected. Patients are requested to provide archival tissues from a prior biopsy or surgery that is treatment-naïve including prior 1) chemotherapy, radiation and 2) anti PD(L)-1 treatment-naïve, pending availability. An on-treatment biopsy is required when medically feasible, after the third dose of the study treatment, treatment week 6. For patients who progress as determined per RECIST (Response evaluation criteria in solid tumors) 1.1/iRECIST criteria, an optional biopsy will be obtained at the time of disease progression.

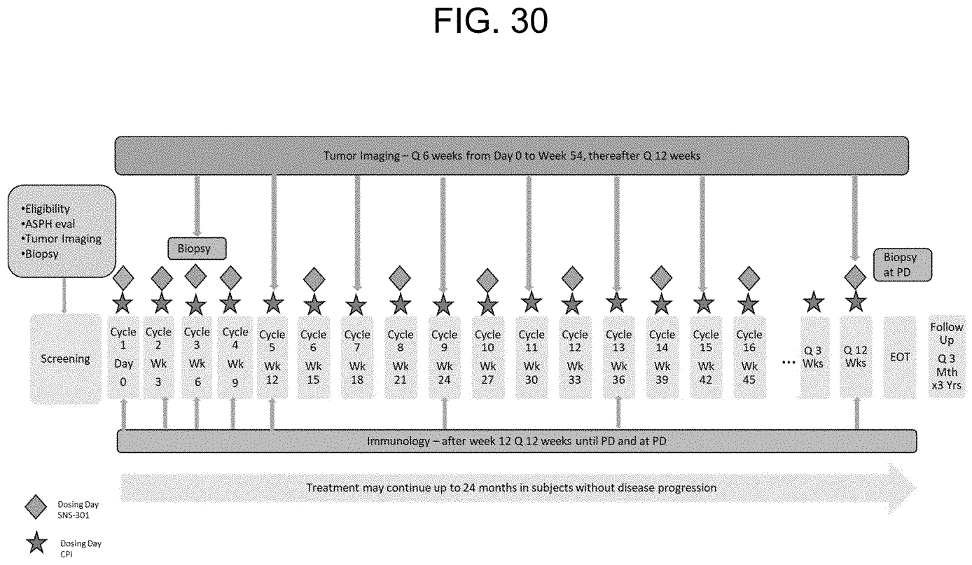

The following procedures will be performed. A schematic of the procedures is provided in FIG. 30.

1. For eligible patients, the study treatment of SNS-301 in addition to pembrolizumab will be initiated on Day 0 (First dose). SNS-301 will be dosed approximately 1 hour after IV infusion of pembrolizumab for the first dose. Subsequent doses of SNS-301 and pembrolizumab can be dosed in any order. Treatment with SNS-301 vaccine therapy may continue if pembrolizumab is discontinued by the Investigator prior to 24 months.

2. Tumor biopsies will be collected at the time of screening, at Week 6 (±3 days) and at first evidence of radiographic or clinical disease progression if clinically deemed feasible. Patients who are unable to undergo biopsy sample collection during treatment but otherwise meet criteria listed in the protocol may continue to receive study treatment.

3. SNS-301 will be administered intradermally using the 3M micro-needle device every 3 weeks (±3 days) for 4 doses then every 6 weeks (±3 days) for 6 additional doses, thereafter every 12 weeks (±3 days) until confirmed disease progression, unacceptable toxicity, deemed intolerable by the investigator or up to 24 months in patients without disease progression.

4. Pembrolizumab will be administered every 3 weeks until confirmed disease progression, unacceptable toxicity, deemed intolerable by the investigator or up to 24 months in patients without disease progression.

5. Imaging will be performed at 6 weeks (±7 days) calculated from the first dose and will continue to be performed every 6 weeks (±7 days), for the first 54 weeks, or earlier if clinically indicated. Thereafter, imaging will be performed approximately every 12 weeks (±7 days). Imaging timing should follow calendar days and should not be adjusted for delays or changes in treatment administration dates.

6. In patients who discontinue trial therapy for any reason other than radiologically defined confirmed progression, tumor imaging should be performed at the time of treatment discontinuation (±4 weeks). If previous tumor imaging was obtained within 4 weeks prior to the date of discontinuation, then additional tumor imaging at treatment discontinuation is not required.

7. Patients will be followed for all adverse events (AEs) for 30 days and for adverse events of special interest (AESI) and serious adverse events (SAEs) occurring up until 90 days after the last dose of study treatment or until the start of a new anti-cancer treatment, whichever comes first. If the investigator becomes aware of an AESI or SAE that is considered related to study treatment after discontinuation from the trial, those events should be reported to the Sponsor within 24 hours.

8. All patients who experience disease progression, have unacceptable toxicity or start a new anti-cancer therapy and are discontinued from the trial will be followed for survival and subsequent anti-cancer therapy. Patients will be contacted (i.e. by telephone) every 3 months to assess for survival status for up to 3 years, until death or patient withdraws consent.

9. Patients who discontinue from study treatment for reasons other than disease progression (e.g., toxicity) will continue scheduled tumor assessment until disease progression, withdrawal of consent, or start of new anti-cancer therapy, death, or trial termination by sponsor, whichever occurs first.

Study treatments include:

1. SNS-301 (1×10¹¹ dose/1ml) intradermal injection using the 3M® hollow microstructured transdermal system (hMTS) device will be administered every 3 weeks (±3 days) for 4 doses then every 6 weeks (±3 days) for 6 additional doses, and thereafter every 12 weeks (±3 days) until confirmed disease progression, unacceptable toxicity, deemed intolerable by investigator or up to 24 months in patients without disease progression.

2. Pembrolizumab (200 mg dose) IV infusion will be administered over 30 minutes every 3 weeks until confirmed disease progression, unacceptable toxicity, deemed intolerable by investigator or up to 24 months in patients without disease progression.

### Statistical Methods

Approximately 15 participants will be enrolled in Stage 1 and an additional approximately 15 participants will be enrolled in Stage 2, if the cohort is expanded.

To evaluate the primary endpoint of objective response per iRECIST at 12 weeks with a null hypothesis of an objective response rate (ORR) of 5% and an alternative hypothesis of an ORR of 18%, 30 patients in a two-stage design with 15 patients in the first stage and 15 patients in the second stage will be enrolled. Patients with evidence of disease progression or deemed unevaluable at 12 weeks will not be counted towards assessment of futility. At the first stage analysis if at least 1 response is observed out of 15 patients, the study will continue through the second stage. At the second stage analysis, if at least 4 responses are observed out of 30 total patients, the null hypothesis will be rejected, and further research considered warranted. The overall power for objective response rate at 12 weeks is 80%. The overall type I error, the chance of incorrectly rejecting the null hypothesis is 6% (targeted alpha of 0.10). The probability of stopping at the first stage under the null hypothesis is 46%. The operating characteristics of this design are calculated using the exact binomial distribution.

Analysis Populations:

Safety Analysis Set:

The safety analysis will be based on the Safety Analysis Set, which comprises all patients who receive at least 1 dose of the study treatment or component of the study treatment.

Efficacy-Evaluable Analysis Set:

The primary efficacy analysis will be based on the Efficacy-Evaluable Analysis Set, which comprises all patients who receive at least 1 dose of the study treatment or component of the study treatment and have a post baseline response assessment per iRECIST at Week 12. Patients who discontinued prior to Week 12 due to disease progression will be included. Patients who do not have a post baseline response assessment conducted will not be included in the analysis of efficacy.

Safety Run-In Set:

All patients who receive at least 1 dose of the study treatment or component of the study treatment apart of the safety run-in.

Immunologic Analysis Set:

All patients who receive at least 1 dose of the study treatment or component of the study treatment and have at least one valid post-baseline immunologic assessment available.

General Methods:

For continuous variables, descriptive statistics (number (n), mean, median, standard deviation, minimum and maximum) will be presented. For categorical variables, frequencies and percentages will be presented. For time-to-event variables, percentages of patients experiencing that event will be presented and median time-to-event will be estimated using the Kaplan-Meier method. As appropriate, a 95% CI will be presented. Graphical displays will be presented, as appropriate. All data collected will be presented in by-patient data listings.

Patients demographic characteristics including age, gender, and race will be analyzed, with categorical variables summarized in frequency tables while continuous variables summarized using mean (standard deviation) and median (range).

Safety evaluations will be based on the incidence, severity, attribution and type of AEs, and changes in the patient's vital signs, and clinical laboratory results. Summarization of toxicity data will focus on incidence of any serious adverse events, adverse events, drug-related adverse events, and adverse events leading to discontinuation or death, and will be presented in tabular form by system organ class and preferred term. Adverse events will be assessed for severity according to the NCI CTCAE, version 5.0.

Objective response rate (ORR) is defined as the proportion of patients with a confirmed best response of CR or PR by RECIST 1.1. Objective response rate will be estimated, and 95% CI based on the exact binomial distribution will be presented.

### Inclusion Criteria

In order to be eligible for participation in this trial, the patient must:

1. Provide signed IRB approved informed consent in accordance with institutional guidelines.

2. Be 18 years of age or older on the day of signing the informed consent, and able and willing to comply with all trial procedures.

3. Have histologically or cytologically documented locally advanced unresectable or metastatic/recurrent ASPH+ SCCHN and currently receiving pembrolizumab or nivolumab.

a. Eligible patients currently receiving pembrolizumab or nivolumab must be considered by Investigator to have the potential to derive clinical benefit from continued treatment with pembrolizumab.

b. Based on RECIST 1.1/iRECIST criteria on current pembrolizumab or nivolumab treatment (prior to initiation of this study), patients must have a best response of stable disease (SD) or first evidence of progressive disease (PD) after a minimum of 12 weeks of pembrolizumab or nivolumab therapy.

c. Patients receiving first-line pembrolizumab monotherapy prior to this study must be PD-L1 positive.

d. Patients on nivolumab therapy must be willing to switch over to pembrolizumab therapy.

4. Have demonstrated intra-tumoral ASPH expression by IHC.

5. Have measurable disease, as defined by RECIST version 1.1 (investigator assessment).

6. Have a performance status of 0 or 1 on Eastern Cooperative Oncology Group (ECOG) Performance Scale.

7. Have a life expectancy of ≥3 months.

8. Be willing to provide a pre-treatment tissue sample obtained after initiation of ongoing pembrolizumab or nivolumab therapy and first dose of SNS-301 and pembrolizumab on this current clinical trial unless clinically contra-indicated per treating physician. Patients unable to provide pre-treatment biopsy while on CPI will be evaluated on a case-by-case basis for enrollment pending Sponsor consultation. Patients are requested to also provide archival tissue from a prior biopsy or surgery that is treatment-naïve including prior 1) chemotherapy, radiation and/or 2) anti-PD(L)-1 treatment-naïve, pending availability. Tissue provided pre-treatment (fresh or archival) will be used to determine ASPH expression and eligibility for the trial. Additionally, an on-treatment biopsy is required unless clinically contraindicated, after the third dose of study treatment at week 6. For patients who progress as determined per RECIST1.1/iRECIST criteria, an optional biopsy will be obtained at the time of disease progression.

9. Have an ECG with no clinically significant findings such as stage 2b and 3 heart block, any history of ventricular arrhythmias or NYHA heart failure within the past 6 months, and QTc prolongation >500 ms or as deemed clinically significant by the investigator and performed within 28 days prior to first dose.

10. Demonstrate adequate organ function: hematological, renal, hepatic, coagulation parameters as defined below and obtained within 28 days prior to the first study treatment. Adequate hematologic and end-organ function must be demonstrated.

### Study Objectives

### Primary Objectives

To determine the safety and tolerability of SNS-301 delivered by intradermal injection (ID) using the 3M® hollow microstructured transdermal system (hMTS) device in addition to pembrolizumab among patients with ASPH+ locally advanced unresectable or metastatic/recurrent SCCHN.

To evaluate the anti-tumor activity of SNS-301 delivered by intradermal injection (ID) using the 3M® hollow microstructured transdermal system (hMTS) device in addition to pembrolizumab in patients with ASPH+ locally advanced unresectable or metastatic/recurrent SCCHN

### Secondary Objective

To evaluate preliminary immune response to SNS-301 delivered by intradermal injection (ID) using the 3M® hollow microstructured transdermal system (hMTS) device in addition to pembrolizumab in patients with ASPH+ locally advanced unresectable or metastatic/recurrent SCCHN.

### Exploratory Objective

To evaluate tumor and immune biomarkers and their association with treatment outcome (antitumor activity and/or safety) in ASPH+ patients with locally advanced unresectable or metastatic/recurrent SCCHN.

### Study Endpoints

### Primary

1. To determine the safety and tolerability of SNS-301 delivered by intradermal injection (ID) using the 3M® hollow microstructured transdermal system (hMTS) device in addition to pembrolizumab among patients with ASPH+ locally advanced unresectable or metastatic/recurrent SCCHN as measured by the following parameters:

All adverse events (AEs) by CTCAE v5 such as clinically significant changes in safety laboratory parameters from baseline: CBC with Differential; Chemistry Panel; Urinalysis; T3, Free T4 and TSH; creatine phosphokinase (CPK) and including adverse events of special interest (AESI) classified by system organ class (SOC), preferred term (PT), severity and relationship to drug

2. To determine anti-tumor activity of SNS-301 delivered by intradermal injection (ID) using the 3M® hollow microstructured transdermal system (hMTS) device in addition to pembrolizumab in patients with ASPH+ locally advanced unresectable or metastatic/recurrent SCCHN as measured by the following parameters: ⋅ Objective response rate (ORR) by immune Response Evaluation Criteria in Solid Tumors (iRECIST)Duration of Response (DoR) as assessed by RECIST version 1.1 and iRECIST
Objective response rate (ORR) by Response Evaluation Criteria (RECIST) version 1.1 by investigator review
Disease control rate (DCR) as assessed by RECIST version 1.1 and iRECIST
Progression Free Survival (PFS) as assessed by RECIST version 1.1 and iRECIST
Overall Survival (OS)

### Secondary

To determine preliminary immune response to SNS-301 delivered by intradermal injection (ID) using the 3M® hollow microstructured transdermal system (hMTS) device in addition to with pembrolizumab in patients with ASPH+ locally advanced unresectable or metastatic/recurrent SCCHN by the following parameters:
Antigen-specific cellular immune responses that may be assessed by but not limited to: Interferon-γ secreting T lymphocytes in peripheral blood mononuclear cells (PBMCs) by ELI Spot
T-cell activation and cytolytic cell phenotype in PBMCs by Flow Cytometry or secretion of immune molecules
B cell activation/antibody secretion
Assessment of Myeloid Derived Suppressor Cells (MDSC)
TCR sequencing of PBMCs for diversity and putative antigen specificity
Immune gene transcript profiling of PBMCs
Assessment of pro-inflammatory and immunosuppressive elements in neoplastic and adjacent normal tissue, where feasible

### Exploratory

To determine tumor and immune biomarkers and their association with treatment outcome (antitumor activity and/or safety) in ASPH+ patients with locally advanced unresectable or metastatic/recurrent solid tumors as measured by the following parameters:
Immune related gene expression to predict treatment efficacy evaluating pre and post-treatment peripheral blood samples and pre-and post-treatment tumor tissue
Expression of tumor specific oncoproteins including but not limited to ASPH
Correlation of serum ASPH level as determined by ELISA with tissue expression using IHC
miRNA profiling to predict treatment efficacy evaluating pre and post-treatment peripheral blood samples as well as urine samples
Cytokine and chemokine profiles in urine pre- and post-treatment and longitudinally throughout the trial
CtDNA analysis and tracking for progression

### Efficacy Assessments

### Tumor Assessment

Initial (screening) tumor assessments must be performed within 28 days prior to the first dose of study treatment. The investigator/site radiologist must review pre-trial images to confirm the patient has measurable disease per RECIST 1.1. Tumor assessments performed as standard of care prior to obtaining informed consent and within 28 days of the first dose of study treatment may be used rather than repeating tests Beginning with screening, all imaging assessments will be evaluated using RECIST 1.1. On-study imaging assessments will be performed every 6 weeks (Q6W) calculated from the date of therapy initiation and independent of treatment delays. RECIST 1.1 will be used by the site for treatment decisions until the first radiologic evidence of progressive disease (PD).

Following the first radiologic evidence of PD by RECIST 1.1, treatment decisions may be made by using immune iRECIST to accommodate tumor response patterns seen with checkpoint inhibitor therapy including pembrolizumab treatment (e.g., tumor flare). This was described by Nishino, et al. 2016 [12] and is used in immunotherapy clinical trials. For a clinically stable subject with first radiologic evidence of PD, it is at the discretion of the site investigator to continue treating the subject with SNS-301 and pembrolizumab until PD is confirmed at least 4 weeks after the date of the first tumor imaging suggesting PD per the site investigator. If radiologic PD is confirmed by the subsequent tumor imaging, the subject should be discontinued from treatment unless, in the opinion of the investigator, the subject is achieving a clinically meaningful benefit. In this case, an exception for continued treatment may be considered following consultation with the sponsor. Additional treatment response evaluation by RECIST v 1.1 and iRECIST may be performed at the sponsor's discretion.

Patients will undergo tumor assessments every 6 weeks (±7 days) for the first 54 weeks (approximately 12 months) following first dose of study treatment, or earlier if clinically indicated. After 54 weeks, tumor assessments will be required every 12 weeks (±7 days). Imaging should continue to be performed until disease progression is assessed by the Investigator, the start of new anti-cancer treatment, withdrawal of consent, death or the end of the trial, whichever occurs first for efficacy follow-up. Patients who start a new anti-cancer therapy will be censored for survival and progression analyses at date of last scan prior to the start of new anti-cancer therapy.

Tumor imaging should be performed by computed tomography (CT), but may be performed by magnetic resonance imaging (MRI) if CT is contraindicated, but the same imaging technique should be used in patient throughout the trial. CT scans (with oral/IV contrast unless contraindicated) must include chest, abdomen and pelvis. The investigator must review before dosing at the next visit. Per RECIST 1.1, response should be confirmed by a repeat radiographic assessment. The scan for confirmation of response may be performed no earlier than 4 weeks after the first indication of response, or at the next scheduled scan, whichever is clinically indicated.

Patients who have unconfirmed disease progression may continue on treatment until progression is confirmed. If radiologic imaging by local/site assessment shows progressive disease (PD), tumor assessment may be repeated 4 weeks later in order to confirm PD with the option of continuing treatment per below while awaiting radiologic confirmation of progression. If repeat imaging shows SD, PR or CR, treatment may be continued as per treatment schedule. If repeat imaging still meets the threshold for PD (≥20% increase in tumor burden compared to nadir) but shows a reduction in tumor burden compared to the previous time point, treatment may be continued as per treatment calendar after consultation with sponsor. If repeat imaging confirms progressive disease without reduction in tumor burden compared to the previous time point, patients will be discontinued from study treatment.

The decision to continue study treatment after the first evidence of disease progression is at the investigator's discretion based on the clinical status of the patient as described in Table 2 below. Confirmatory imaging may be performed as early as 28 days later; alternatively, the scan performed at the next scheduled time point may be used as confirmation. Patients may receive study treatment while waiting for confirmation of PD if they are clinically stable as defined by the following criteria:
Absence of signs and symptoms (including worsening of laboratory values) indicating disease progression
No decline in ECOG performance status from baseline
Absence of rapid progression of disease
Absence of progressive tumor at critical anatomical sites (e.g., cord compression) requiring urgent alternative medical intervention
Evidence of clinical benefit (defined as the stabilization or improvement of disease-related symptoms) as assessed by the investigator

TABLE 2
Imaging and Treatment after First Radiologic Evidence of Progressive Disease

| | Clinically Stable | | Clinically Unstable | |
|---|---|---|---|---|
| | Imaging | Treatment | Imaging | Treatment |
| 1st radiologic | Repeat imaging | May continue | Repeat imaging | Discontinue |
| evidence of PD | at ≥4 weeks at | study treatment | at ≥4 weeks at | treatment |
| | site to confirm | at the | site to confirm | |
| | PD | Investigator's | PD per physician | |
| | | discretion while | discretion only | |
| | | awaiting | | |
| | | confirmatory | | |
| | | scan by site | | |
| Repeat scan | No additional | Discontinue | No additional | N/A |
| confirms PD (no | imaging required | treatment | imaging required | |
| reduction in | | | | |
| tumor burden | | | | |
| from prior scan) | | | | |
| Repeat scan | Continue | Continue study | Continue | May restart |
| confirms PD | regularly | treatment after | regularly | study treatment |
| (reduction in | scheduled | consultation | scheduled | if condition has |
| tumor burden | imaging | with Sponsor | imaging | improved and/or |
| from prior scan) | assessments | | assessments | clinically stable |
| | | | | per Investigator |
| | | | | and Sponsor's |
| | | | | discretion |
| Repeat scan | Continue | Continue study | Continue | May restart |
| shows SD, PR or | regularly | treatment at the | regularly | study treatment |
| CR | scheduled | Investigator's | scheduled | if condition has |
| | imaging | discretion | imaging | improved and/or |
| | assessments | | assessments | clinically stable |
| | | | | per |
| | | | | Investigator's |
| | | | | discretion |

### Safety Assessments

### Demographics and Medical History

Demographics will include gender, year of birth, race and ethnicity.

Medical history will include details regarding the patients overall medical and surgical history as well as detailed information regarding the patient's previous treatment, including systemic treatments, radiation and surgeries, pathology, risk stratification, etc. since their original diagnosis. HPV status, EBV status and progression data will also be collected. Reproductive status and smoking/alcohol history will also be captured. PD-L1 status will also be collected, if available.

### Physical Examinations

A complete physical exam will include, at a minimum head, eyes, ears, nose, throat and cardiovascular, dermatological, musculoskeletal, respiratory, gastrointestinal and neurological systems. Height (screening only) and weight will also be collected. Additionally, any signs and symptoms, other than those associated with a definitive diagnosis, should be collected at baseline and during the study.

During the study, a targeted, symptom-directed exam, as clinically indicated will be performed within 72 hours of each dosing visit

### Eastern Cooperative Oncology Performance Status

The health, activity and well-being of the patient will be measured by the ECOG performance status and will be assessed on a scale of 0 to 5 with 0 being fully active and 5 being dead. ECOG performance status will be collected within 72 hours of each dosing visit.

### Vital Signs

Vital signs will include temperature, blood pressure, pulse rate and respiratory rate. For first infusion of pembrolizumab, the patient's vital signs should be determined within 60 minutes before the infusion. If clinically indicated, vital signs should be recorded at 15, 30, 45, and 60 minutes (±5 minutes for all timepoints) after the start of the infusion, and 30 (±10) minutes after the infusion. For subsequent infusions, vital signs will be collected within 60 minutes before the infusion and at 30 (±5) minutes after the infusion. Patients will be informed about the possibility of delayed post-infusion symptoms and instructed to contact their trial physician if they develop such symptoms.

### Electrocardiograms

A 12-lead ECG will be obtained at screening and when clinically indicated. Patients should be resting in a supine position for at least 10 minutes prior to ECG collection.

### Clinical Safety Laboratory Assessments

Hematological toxicities will be assessed in term of hemoglobin value, white blood cell, neutrophil, platelet and, lymphocyte count according to NCI-CTCAE V5.0 AE grading.

Laboratory abnormalities (grade 1 and greater that are listed in the NCI-CTCAE V5.0) should be recorded on the AE page regardless of their causality. Laboratory abnormalities associated with a definitive diagnosis will not be recorded as and AE unless it has become worse since baseline. Test analytes are provided Table 3 below.

Safety labs will be performed within 72 hours of each dosing visit.

TABLE 3
Test Analytes

| | |
|---|---|
| Hematology | Serum chemistry |
| Hematocrit (Hct) | Albumin |
| Hemoglobin (Hgb) | Alanine aminotransferase (ALT) |
| Platelet count | Aspartate aminotransferase (AST) |
| Red blood cell (RBC) count | Alkaline phosphatase (ALP) |
| White blood cell (WBC) count | Blood Urea Nitrogen (BUN) or Urea |
| Neutrophils | Bicarbonate or Carbon dioxide (CO2) |
| Lymphocytes | Creatinine |
| Eosinophils | Creatine phosphokinase (CPK) |
| Monocytes | Electrolytes (Na, K, Mg, Cl, Ca, P) |
| Basophils | Glucose (either fasting or non-fasting) |
| Other cells, if any | Lactate dehydrogenase (LDH) |
| Platelets | Total bilirubin (direct bilirubin if elevated) |
| Thyroid | Total protein |
| TSH, T3 and FT4 | Urinalysis |
| Coagulation | Specific gravity |
| International normalized ratio/INR | pH |
| Activated partial thromboplastin time (PTT) | Glucose |
| Other anticoagulant monitoring (if required) | Protein |
| HIV screen (at screening, if indicated) | Ketones |
| Hepatitis screen (at screening, if indicated) | Blood |
| HPV/EBV screen (at screening, if unknown) | Microscopic exam, if abnormalities |
| Pregnancy test | |

### Hepatitis and HIV Screening

Patients should be tested for HIV locally prior to the inclusion into the trial only based on investigator's clinical suspicion for HIV infection and HIV-positive patients will be excluded from the clinical trial. Hepatitis B surface antigen, anti-HBc antibody, anti-HBs antibody, and Hepatitis C antibody immunoassays should be tested only per investigator's clinical suspicion during screening and tested locally. In patients who have positive serology for the anti-HBc antibody, HBV DNA should be tested prior to Day 0.

### Pregnancy Test

A Serum pregnancy test (for women of childbearing potential, including women who have had a tubal ligation) must be performed and documented as negative within 72 hours prior to each dose.

### Urinalysis

Urinalysis includes specific gravity, pH, glucose, protein, ketones, blood, and a microscopic exam if abnormal results are noted.

### TSH, T3 and FT4

Thyroid function tests will be performed at screening and every 6 weeks thereafter.

### Creatine Phosphokinase (CPK)

CPK will be performed at screening and at the discontinuation visit.

### HPV/EBV Testing

If the patient's HPV/EBV status is unknown, they should be tested prior to receiving SNS-301. The results do not need to be known before the patient receives study treatment.

### Immunogenicity Assessments

Urine

Urine samples will be obtained for biomarker evaluation. Samples may be tested for the presence and level of various cytokines by ELISA which may be indicative of activated immune responses. Samples may also be tested by ELISA for the presence and level of ASPH and/or other cancer biomarkers which may be indicative of cancer status. Samples may also be processed to obtain tumor cells (and their derivatives) for further determination and analysis of cancer status. miRNA profiling of pre and post-treatment urine samples may also be performed to predict treatment efficacy.

Blood Assays

Blood assays include those measured in serum, plasma and whole blood/PBMCs.

Serum and plasma

Serum and plasma are collected for the direct measure of ASPH levels, anti-ASPH antibodies, anti-phage antibodies and other tumor biomarkers.

ASPH

Subject sera and/or plasma will be tested for the presence of ASPH and/or exosomes that contain ASPH on their surface by ELISA using several different monoclonal antibodies that are reactive with the ASPH protein. The presence of ASPH in serum or plasma is an indicator of cancer status. Alterations in ASPH levels may be indicative of response to treatment.

Anti-ASPH antibodies

Production of anti-ASPH antibodies is a direct result of an active immune response to the vaccine. Levels of anti-ASPH antibody are expected to rise during an active immune response and should reach a plateau level at maximal response. Continued and regular boosting of the vaccine during the course of treatment is expected to maintain or restore this level of anti-ASPH antibody in serum.

Anti-phage antibodies

Because the vaccine is delivered using a bacteriophage vector, production of anti-phage antibodies is also expected and is a direct result of an active immune response to the vaccine. High levels of anti-phage antibody may result in neutralization of further doses/boosts of vaccine. During the Phase I clinical study it was found that the use of a lower dose of vaccine during initial vaccination attenuate the production of anti-phage antibodies and this finding contributed to the selection of the dose for the current trial. Levels of anti-phage antibodies will be monitored here to ascertain if any correlation exists between the production of anti-phage antibodies and reduced efficacy of the vaccine.

Other tumor and immune biomarkers

Levels of other cancer biomarkers and cytokines may also be tested in serum and/or plasma and may also be used to monitor cancer status and response to treatment. miRNA profiling of pre and post-treatment serum and/or plasma samples may also be performed to predict treatment efficacy.

Circulating tumor DNA (ctDNA)

Blood samples will be collected in Streck tubes for isolation of ctDNA. ctDNA analysis may be used as a tool to monitor for treatment efficacy and resistance and for predicting the likelihood of relapse.

Whole blood/peripheral blood mononuclear cells (PBMCs)

PBMCs are collected to monitor overall and specific immune responses.

Immunophenotyping

Immunophenotyping will be performed by flow cytometry to monitor the levels of all immune cells including B-cells, CD4⁺ T-cells, CD8⁺ T-cells, NK cells, monocytes, neutrophils, eosinophils and myeloid derived suppressor cells (MDSCs). In patients mounting an active immune response it is expected for the percentages of certain cell types to increase.

Gene transcript signatures from PBMCs to assess the profile of immune-related gene transcripts may be performed on PBMCs with or without prior in vitro stimulation.

B-cells

B-cells form the humoral (antibody) response arm of the immune system. Vaccination with SNS-301 is expected to result in maturation of anti-ASPH specific B-cells.

B-cell profiling

As B-cells mature they transition through multiple stages that are distinguishable by the analysis of the presence or absence of specific surface antigens. Percentages of naïve B-cells, transitional B-cells, activated B-cells, plasmablasts, plasma cells and memory B-cells will be determined by multi-parameter flow cytometry.

ASPH-specific B-cells

ASPH-specific B-cells are a direct measure of the immune response to the SNS-301 vaccine. Flow cytometry will be used to determine the changes in the levels of ASPH-specific B-cells. Furthermore, these B-cells may be isolated, cloned and expanded ex vivo and the resulting anti-ASPH antibodies characterized via epitope mapping.

T-cells

T-cells form the cellular arm of the immune response. Vaccination with SNS-301 is expected to result in maturation and activation of ASPH specific T-cells.

T-cell profiling

The cellular immune response can generally be characterized as having to primary arms, CD4⁺ helper T-cell responses and CD8⁺ cytotoxic T-cell responses. In preclinical studies as well as the phase I clinical trial of SNS-301, activation of both T-cell subsets was noted. Furthermore, immune responses are often hampered by the presence of regulatory T-cells which may downregulate T-cell responses. Multi-parameter flow cytometry will be used to characterize the various subsets of T-cells in peripheral blood during the entire course of the study. Flow cytometric assays will also be utilized to assess the presence of cells that are known to play a role in immune suppression and may include an examination of the influence of these cells on the induction or expansion of an immune response after immunotherapy. Markers that may be used for this purpose include CD3, CD16, CD19, CD20, CD56, CD11b, CD14, CD15, CD33 and HLA-DR. These markers may change relative to new data becoming available that is informative for this assessment.

ASPH-specific T-cells

T cell responses will be assessed using antigen-specific IFN-γ ELISpot assay using antigen presenting cells loaded with either full-length recombinant ASPH protein or overlapping peptide libraries covering the SNS-301 antigens. Antigen specific T cell responses will also be assessed via flow cytometry. Flow cytometric assays may include an examination of the influence of immunotherapy on the ability of patient T cells to exhibit phenotypic markers associated with cytolytic potential, activation or exhaustion after stimulation by peptides corresponding to SNS-301 antigens. Markers that may be used for this purpose include CD3, CD4, CD8, CD137, CD69, CD38, PD1, Granzyme A, Granzyme B and Perforin. These markers may change relative to new data becoming available that is informative for this assessment. Additionally, T-cell responses to general immune stimulators may be evaluated in order to track general cellular immune competence during the trial.

Additionally, ASPH-specific T-cells may be isolated, cloned and expanded ex vivo. For expansion antigen presenting cells loaded with either full-length recombinant ASPH protein or overlapping peptide libraries covering the SNS-301 antigens would be employed. These T-cells may be characterized by sequencing of their T-cell receptors (TCRs) to assess diversity and putative antigen specificity.

Tissue

A tumor specimen obtained after completion of the most recent systemic therapy should be submitted. In patients undergoing a pre-treatment biopsy, an archival tumor specimen, if available, should also be submitted. After signing of the Informed Consent Form, tumor tissue should be submitted to the Sponsor in a timely manner. All patients will undergo a mandatory tumor biopsy sample collection, if clinically feasible as determined by the trial investigator in consenting patients, at Week 6/3rd dose (+/−3 days) and at the time of first evidence of radiographic or clinical disease progression. For patients who respond and subsequently progress, an optional biopsy may be obtained at the time of disease progression. Tumor tissue should be of good quality based on total and viable tumor content. Acceptable samples include core needle biopsies for deep tumor tissue or lymph nodes or excisional, incisional, punch, or forceps biopsies for cutaneous, subcutaneous, or mucosal lesions. Fine-needle aspiration may be acceptable pending sponsor approval however, brushing, cell pellets from pleural effusion, and lavage samples are not acceptable. For core needle biopsy specimens, at least three cores should be submitted for evaluation. Patients who are unable to undergo biopsy sample collection but otherwise meet criteria outlined in protocol may continue to receive study treatment.

If a tumor biopsy is to be obtained from an intended target lesion during eligibility assessment, the biopsy should be performed prior to obtaining the baseline scan. Otherwise, a new baseline scan should be obtained.

Archival and fresh tumor tissue samples should be representative tumor specimens in formalin-fixed paraffin embedded (FFPE) blocks (preferred) or at least 15 unstained slides, with an associated pathology report, should be submitted for intra-tumoral immunology assessments. Tissue slices of 4-5 microns are mounted on positively charged glass slides. Slides should be unbaked and stored cold or frozen.

Tissue Assays

Available tumor tissue collected from pre- and post- treatment may be assessed for the presence of immune cells using immunohistochemistry or immunofluorescence. The presence of immune signatures may also be analyzed through the assessment of various transcripts suggestive of an inflammatory or an immunosuppressive tissue microenvironment.

Tumor tissue will be collected for immunology assessments including but not limited to markers related to inflammation, suppression, T cell infiltration, and associated tumor microenvironment characteristics. Tumor infiltrating lymphocytes may be isolated and subjected to single cell expression profiling and/or TCR sequencing.

In addition, exploratory biomarkers may be evaluated.

ASPH Immunohistochemistry (IHC) Assay

ASPH testing will be done by immunohistochemistry on either fresh or archival tumor tissue. Tissue will be deparaffinized and rehydrated, quenched with hydrogen peroxide and blocked with horse serum. Slides are stained overnight at 4° C. with an ASPH-specific murine monoclonal or a non-relevant mouse IgG as a negative control. Detection employs a secondary anti-mouse antibody and a chromogenic substrate. Slides are counterstained with hematoxylin and cover slipped. Semiquantitative analysis of staining intensity and distribution of ASPH levels is evaluated according to the following scale (0, negative; 1+, moderate; 2+, strong; and 3+, very strong immunoreactivity).

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the disclosure have been illustrated and described, various other changes and modifications can be made without departing from the spirit and scope of the disclosure. The scope of the appended claims includes all such changes and modifications that are within the scope of this disclosure.

### Numbered Embodiments

Notwithstanding the appended claims, the disclosure sets forth the following numbered embodiments:

1. A method for inhibiting growth and/or proliferation of cancer cells in a subject, the method comprising administering to the subject an effective amount of a composition comprising a bacteriophage expressing a fragment of human aspartate β-hydroxylase (ASPH) and an effective amount of an immune checkpoint protein inhibitor.

2. The method of embodiment 1, wherein the cancer cells are prostate, liver, bile duct, brain, head-and-neck, breast, colon, ovarian, cervical, pancreatic or lung cancer cells.

3. The method of embodiment 1 or 2, wherein the cancer cells express human ASPH.

4. A method for treating or ameliorating cancer or a symptom of cancer in a subject, the method comprising administering to the subject an effective amount of a composition comprising a bacteriophage expressing a fragment of human ASPH and an effective amount of an immune checkpoint protein inhibitor.

5. The method of embodiment 4, wherein the cancer is prostate, liver, bile duct, brain, head-and-neck, breast, colon, ovarian, cervical, pancreatic or lung cancer.

6. The method of embodiment 5, wherein the cancer is ASPH-positive squamous cell cancer of the head and neck (SCCHN).

7. The method of embodiment 6, wherein the SCCHN is locally advanced unresectable SCCHN, metastatic SCCHN or recurrent SCCHN.

8. The method of embodiment 4, wherein the cancer is a hematologic malignancy.

9. The method of any one of embodiments 4-8, wherein the cancer is human ASPH-expressing cancer.

10. The method of any one of embodiments 1-9, wherein the bacteriophage is bacteriophage lambda.

11. The method of embodiment 10, wherein the bacteriophage lambda expresses amino acids 113-311 from the N-terminal region of ASPH fused at the C-terminus of the bacteriophage lambda head decoration protein D (gpD).

12. The method of embodiment 10, wherein the bacteriophage lambda expresses a fusion protein comprising, in N-terminus to C-terminus order, (1) a gpD fragment, (2) a linker sequence and (3) a fragment of human ASPH.

13. The method of embodiment 12, wherein the gpD fragment is the amino acid sequence of SEQ ID NO: 2.

14. The method of embodiment 12 or 13, wherein the linker sequence comprises SEQ ID NO: 3.

15. The method of any one of embodiments 12-14, wherein the fragment of human ASPH consists of SEQ ID NO: 4.

16. The method of embodiment 10, wherein the bacteriophage lambda expresses a protein comprising or consisting of the amino acid sequence of SEQ ID NO: 1.

17. The method of any one of embodiments 1-16, wherein the composition comprising a bacteriophage is administered at a dose of about 1×10¹¹ particles in a 1 ml intradermal injection

18. The method of embodiment 17, wherein the composition comprising a bacteriophage is administered every 3 weeks ±3 days for 4 doses, then every 6 weeks ±3 days for 6 additional doses, and thereafter every 12 weeks ±3 days for up to 24 months.

19. The method of any one of embodiments 1-18, wherein the immune checkpoint protein is Programmed Death-1 (PD-1) or Programmed Death Ligand-1 (PD-L1).

20. The method of any one of embodiments 1-19, wherein the immune checkpoint protein inhibitor disrupts the interaction between PD-1 and PD-L1.

21. The method of any one of embodiments 1-20, wherein the immune checkpoint protein inhibitor is an antibody or an antibody fragment that targets PD-1.

22. The method of embodiment 21, wherein the antibody that targets PD-1 is pembrolizumab.

23. The method of embodiment 22, wherein the pembrolizumab is administered at a dose of about 200 mg as an intravenous infusion over about 30 minutes.

24. The method of embodiment 23, wherein the pembrolizumab is administered about every 3 weeks.

25. The method of any one of embodiments 1-20, wherein the immune checkpoint protein inhibitor is an antibody or an antibody fragment that targets PD-L1.

26. A method for treating or ameliorating cancer or a symptom of cancer in a subject, the method comprising administering to the subject an effective amount of a composition comprising a bacteriophage expressing a fragment of human ASPH and an effective amount of an immune checkpoint protein inhibitor; wherein the composition comprising the bacteriophage comprises bacteriophage lambda expressing a fusion protein comprising or consisting of the amino acid sequence of SEQ ID NO: 1; wherein the composition comprising the bacteriophage is administered at a dose of about 1×10¹¹ particles in a 1 ml intradermal injection every 3 weeks ±3 days for 4 doses then every 6 weeks ±3 days for 6 additional doses, thereafter every 12 weeks ±3 days; and wherein the immune checkpoint protein inhibitor is pembrolizumab, and wherein the pembrolizumab is administered at a dose of about 200 mg as an intravenous infusion over about 30 minutes every 3 weeks.

27. The method of embodiment 26, wherein the cancer is ASPH-positive head-and-neck cancer.

28. A composition comprising: a pharmaceutical composition comprising a bacteriophage expressing a fragment of human aspartate β-hydroxylase (ASPH); and a pharmaceutical composition comprising an immune checkpoint protein inhibitor; wherein said pharmaceutical compositions are in separate containers.

29. The composition of embodiment 28, wherein the bacteriophage is a bacteriophage lambda that expresses a protein comprising or consisting of the amino acid sequence of SEQ ID NO: 1.

30. The composition of embodiment 28 or 29, wherein the immune checkpoint protein is Programmed Death-1 (PD-1) or Programmed Death Ligand-1 (PD-L1).

1. A method for inhibiting growth and/or proliferation of cancer cells in a subject, the method comprising administering to the subject an effective amount of a composition comprising a bacteriophage expressing a fragment of human aspartate β-hydroxylase (ASPH) and an effective amount of an immune checkpoint protein inhibitor.

2. The method of claim 1, wherein the cancer cells are prostate, liver, bile duct, brain, head-and-neck, breast, colon, ovarian, cervical, pancreatic or lung cancer cells.

3. The method of claim 1, wherein the cancer cells express human ASPH.

4. A method for treating or ameliorating cancer or a symptom of cancer in a subject, the method comprising administering to the subject an effective amount of a composition comprising a bacteriophage expressing a fragment of human ASPH and an effective amount of an immune checkpoint protein inhibitor.

5. The method of claim 4, wherein the cancer is prostate, liver, bile duct, brain, head-and-neck, breast, colon, ovarian, cervical, pancreatic or lung cancer.

6. The method of claim 5, wherein the cancer is ASPH-positive squamous cell cancer of the head and neck (SCCHN).

7. The method of claim 6, wherein the SCCHN is locally advanced unresectable SCCHN, metastatic SCCHN or recurrent SCCHN.

8. The method of claim 4, wherein the cancer is a hematologic malignancy.

9. The method of claim 4, wherein the cancer is human ASPH-expressing cancer.

10. The method of claim 1, wherein the bacteriophage is bacteriophage lambda.

11. The method of claim 10, wherein the bacteriophage lambda expresses amino acids 113-311 from the N-terminal region of ASPH fused at the C-terminus of the bacteriophage lambda head decoration protein D (gpD).

12. The method of claim 10, wherein the bacteriophage lambda expresses a fusion protein comprising, in N-terminus to C-terminus order, (1) a gpD fragment, (2) a linker sequence and (3) a fragment of human ASPH.

13. The method of claim 12, wherein the gpD fragment is the amino acid sequence of SEQ ID NO: 2.

14. The method of claim 12, wherein the linker sequence comprises SEQ ID NO: 3.

15. The method of claim 12, wherein the fragment of human ASPH consists of SEQ ID NO: 4.

16. The method of claim 10, wherein the bacteriophage lambda expresses a protein comprising or consisting of the amino acid sequence of SEQ ID NO: 1.

17. The method of claim 1, wherein the composition comprising a bacteriophage is administered at a dose of about 1×10¹¹ particles in a 1 ml intradermal injection

18. The method of claim 17, wherein the composition comprising a bacteriophage is administered every 3 weeks ±3 days for 4 doses, then every 6 weeks ±3 days for 6 additional doses, and thereafter every 12 weeks ±3 days for up to 24 months.

19. The method of claim 1, wherein the immune checkpoint protein is Programmed Death-1 (PD-1) or Programmed Death Ligand-1 (PD-L1).

20. The method of claim 1, wherein the immune checkpoint protein inhibitor disrupts the interaction between PD-1 and PD-L1.

21. The method of claim 1, wherein the immune checkpoint protein inhibitor is an antibody or an antibody fragment that targets PD-1.

22. The method of claim 21, wherein the antibody that targets PD-1 is pembrolizumab.

23. The method of claim 22, wherein the pembrolizumab is administered at a dose of about 200 mg as an intravenous infusion over about 30 minutes.

24. The method of claim 23, wherein the pembrolizumab is administered about every 3 weeks.

25. The method of claim 1, wherein the immune checkpoint protein inhibitor is an antibody or an antibody fragment that targets PD-L1.

26. A method for treating or ameliorating cancer or a symptom of cancer in a subject, the method comprising administering to the subject an effective amount of a composition comprising a bacteriophage expressing a fragment of human ASPH and an effective amount of an immune checkpoint protein inhibitor;
wherein the composition comprising the bacteriophage comprises bacteriophage lambda expressing a fusion protein comprising or consisting of the amino acid sequence of SEQ ID NO: 1;
wherein the composition comprising the bacteriophage is administered at a dose of about 1×10¹¹ particles in a 1 ml intradermal injection every 3 weeks ±3 days for 4 doses then every 6 weeks ±3 days for 6 additional doses, thereafter every 12 weeks ±3 days; and
wherein the immune checkpoint protein inhibitor is pembrolizumab, and wherein the pembrolizumab is administered at a dose of about 200 mg as an intravenous infusion over about 30 minutes every 3 weeks.

27. The method of claim 26, wherein the cancer is ASPH-positive head-and-neck cancer.

28. A composition comprising: a pharmaceutical composition comprising a bacteriophage expressing a fragment of human aspartate β-hydroxylase (ASPH); and a pharmaceutical composition comprising an immune checkpoint protein inhibitor; wherein said pharmaceutical compositions are in separate containers.

29. The composition of claim 28, wherein the bacteriophage is a bacteriophage lambda that expresses a protein comprising or consisting of the amino acid sequence of SEQ ID NO: 1.

30. The composition of claim 28, wherein the immune checkpoint protein is Programmed Death-1 (PD-1) or Programmed Death Ligand-1 (PD-L1).
